(19) United States
(12) Patent Application Publication
(10) Pub. No.: US 2014/0024590 A1
(43) Pub. Date: Jan. 23, 2014

(54) KRAS-Variant And Endometriosis

(71) Applicants: Joanne B. Weidhaas, Westport, CT (US); Hugh S. Taylor, Easton, CT (US)

(72) Inventors: Joanne B. Weidhaas, Westport, CT (US); Hugh S. Taylor, Easton, CT (US)

(73) Assignee: YALE UNIVERSITY, New Haven, CT (US)

(21) Appl. No.: 13/984,918

(22) PCT Filed: Feb. 17, 2012
(86) PCT No.: PCT/US12/25637

§ 371 Date: Oct. 1, 2013

Related U.S. Application Data
(60) Provisional application No. 61/444,292, filed on Feb. 18, 2011.

(51) Int. Cl. C12Q 1/68

(52) U.S. Cl. CPC C12Q 1/6883
USPC 514/10.2; 435/6.11; 514/9.7

(57) ABSTRACT

The invention provides methods for predicting an increased risk or probability of developing endometriosis in a patient based upon the patient's KRAS variant status.

### RELATED APPLICATIONS

This application claims the benefit of provisional application U.S. Ser. No. 61/444,292, filed Feb. 18, 2011, the contents of which are herein incorporated by reference in their entirety.

### INCORPORATION OF SEQUENCE LISTING

The contents of the text file named “34592-514001WO_ST25.txt,” which was created on Jan. 7, 2012 and is 32.8 KB in size, are hereby incorporated by reference in their entirety.

### FIELD OF THE INVENTION

This invention relates generally to the fields of cancer, reproductive health and molecular biology. The invention provides methods for predicting increased risk of developing endometriosis and for predicting response to treatment for endometriosis and potentially predicting which endometriosis cases will progress to ovarian cancer.

### BACKGROUND OF THE INVENTION

Endometriosis is the number one cause of pelvic pain in women of child bearing age, is a common cause of infertility and is predicted to occur in 7-10% of women. This disease often runs in families, indicating a genetic predisposition of certain individuals to developing endometriosis later in life.

Currently there are no markers, including inherited markers, of risk of developing endometriosis. Diagnosis of this disease is made through surgery only, which is invasive and potentially risky to the health of the patient.

Endometriosis can potentially be prevented through progestin or other commonly used and simple therapies if those persons who are at risk of developing the disease are identified prior to presentation of signs or symptoms. Additionally, as there are numerous causes of infertility and of pelvic pain, a non-invasive marker of disease would allow specific therapy as well as avoid surgery in those at low risk for endometriosis.

Thus, there is a long felt need in the art for a marker that predicts an individual's risk of developing endometriosis. Particularly valuable are those markers that predict risk at a time when a prophylactic therapy can be administered such that the emergence of the disease is prevented.

### SUMMARY OF THE INVENTION

Endometriosis is a common, benign gynecological disorder, which is a frequent cause of chronic pelvic pain and infertility in 5-15% of reproductive age women. Although studied for many years, the exact pathogenesis as well as etiology of this disease remain unclear. Activation of the KRAS gene may cause de novo formation of endometriosis in mice, however, no activating mutations have been found in the coding region of this gene in human endometriosis.

As a solution to the problem of determining the pathogenesis and etiology of endometriosis, the invention provides an activating mutation in the regulatory regions of KRAS gene causing excessive production of the protein with subsequent activation of the Ras pathway. Specifically, 132 women with endometriosis were evaluated for a newly identified single-nucleotide polymorphism (SNP) in a let-7 miRNA binding site (also known as a let-7 complementary site or LCS) in the 3′UTR of the KRAS gene. This SNP in the LCS6 of the KRAS gene is associated with an increased risk of lung, breast and ovarian cancer. Thirty-one percent of the endometriosis subjects were found to carry this variant KRAS allele compared to only 5.8% of the general population. The presence of this mutation is associated with higher KRAS mRNA and protein levels as well as lower let-7 levels in endometrial stromal cells of women with endometriosis. The presence of this mutation is associated with an increased proliferation rate and increased invasion capacity of these endometrial cells.

The invention provides a novel gene mutation that is associated with up to one third of endometriosis cases. This KRAS mutation represents a new therapeutic target for endometriosis. Furthermore, the KRAS mutation represents a basis of a potential screening method for endometriosis risk as a biomarker for the future development, onset, or severity of disease.

Evidence supporting the hypothesis that KRAS over-expression could cause endometriosis came from a study of KRAS mutations in mouse models leading to endometriosis (Dinulescu D M et al. Nat Med 11: 63-70. 2005). However, no evidence exists suggesting that human endometriosis has been or is associated with KRAS mutations. Others looked for inherited variations in KRAS, but found no evidence that any identified mutation predicted any risk of developing endometriosis (Zhao et al. Molecular Human Reproduction 12(11): 671-676. 2006). Moreover, previous studies failed to find a KRAS-disrupting variation predictive of response to therapy for endometriosis. In fact, Zhao et al. concluded that the risk of endometriosis in women is not influenced by common variations in KRAS.

The invention provides methods of predicting an individual's risk of developing endometriosis based upon the presence of a genetic marker within the 3′ untranslated region (UTR) of the human KRAS gene, referred to as the “KRAS Variant,” the “LCS6 Variant,” or the “LCS6 SNP” which is a SNP located in a binding site for the let-7 family of miRNA. This KRAS-variant has been shown to lead to increased KRAS expression and is the first identified inherited version of KRAS overexpression/misregulation.

The presence of the KRAS-variant is predictive of the response an individual will present to various treatments for endometriosis. Furthermore, the presence of the KRAS variant and presentation of a sign or symptom of endometriosis are either alone, or in combination, predictive of an increased risk of developing ovarian cancer. The KRAS variant is associated or responsible for the development of approximately, one third of all cases of endometriosis. However, this proportion could be higher, e.g. one half. Expressed as a percentage, the KRAS variant is predictive of 20%, 30%, 40%, 50%, 60%, 70%, 80%, 90%, 95%, 100%, or any percentage of endometriosis cases in between.

The KRAS variant is the first genetic marker of endometriosis. Specifically, the presence of the KRAS variant predicts a genetically-distinct form of endometriosis. As the KRAS-variant has been shown to be associated with ovarian cancer risk, and endometriosis is associated with an increased risk of ovarian cancer, KRAS-variant associated endometriosis might be those at highest risk of progression to ovarian cancer.

Specifically, the invention provides a method of predicting the risk of developing endometriosis in a subject, including the steps of: (a) obtaining a sample from the subject; (b) extracting an isolated DNA or RNA sequence including SEQ ID NO: 6, SEQ ID NO: 13, a combination thereof, or a complementary sequence thereof; wherein the presence of SEQ ID NO: 13 in the sample indicates that the subject has an increased risk of developing endometriosis compared to an individual who does not carry a DNA sequence comprising SEQ ID NO: 13.

The subject may have or present a risk factor for developing endometriosis. Exemplary risk factors include, but are not limited to, a first-degree relative with endometriosis, delayed childbearing, dysmenorrhea, pelvic pain, dyspurunia, dysuria, abnormally long menses, müllerian duct anomalies, infertility, aged 15-44, lacks multiple pregnancies, lack of low-dose oral contraceptive usage, and forfeit of regular exercise. The subject may be further at risk for developing ovarian cancer.

The invention also provides a method of predicting the risk of developing ovarian cancer in a subject who has endometriosis, including the steps of: (a) obtaining a sample from the subject, wherein the subject has endometriosis; (b) extracting an isolated DNA or RNA sequence including SEQ ID NO: 6, SEQ ID NO: 13, a combination thereof, or a complementary sequence thereof; wherein the presence of SEQ ID NO: 13 in the sample indicates that the subject has an increased risk of developing ovarian cancer compared to an individual who does not carry a DNA sequence comprising SEQ ID NO: 13.

In one aspect of these methods, the sample is a cell or a fluid. The cell is optionally isolated from the oral mucosa, pleural cavity, the abdominal cavity, the pelvic cavity, a lung, the large intestine, the small intestine, the bladder, an ovary, a fallopian tube, a ligament, the endometrium, the myometrium, the perimetrium, the peritoneum, the uterus, or the cervix of the subject. The fluid is saliva, whole blood, blood plasma, blood serum, buffy coat, lymph fluid, ascites, serous fluid, or urine collected form the subject. Other normal tissues, including toe or finger nail clippings, can be used as a source of sample.

In another aspect of these methods, the endometriosis is a severe form. Alternatively, or in addition, the endometriosis is characterized by the occurrence of endometriomas. An endometrioma indicates the presence of a severe form of endometriosis within the ovary.

The invention further provides a method of determining the responsiveness of a subject to a form of endometriosis treatment, the method including assaying for the presence of a uracil or thymine to guanine transition a position 4 of LCS6 of KRAS, wherein the presence of the transition predicts whether the endometriosis is resistant or responsive to the form of treatment.

The invention provides a method of preventing the onset of endometriosis in a subject including, (a) assaying for the presence of a uracil or thymine to guanine transition a position 4 of LCS6 of KRAS, wherein the presence of the transition indicates that the subject is at an increased risk of developing endometriosis, and (b) administering a treatment to the subject for endometriosis before the subject presents a sign or symptom of the disease, thereby preventing the onset of endometriosis in the subject.

In one aspect of these methods, the form of treatment is hormonal therapy. Exemplary hormonal therapies include, but are not limited to, estrogen, progestin, progesterone, a testosterone derivative, a gonadotrophin releasing hormone agonist or antagonist, an aromatase inhibitor, or any combination thereof. In a particular embodiment, the hormonal therapy is progestin.

### BRIEF DESCRIPTION OF THE DRAWINGS

FIG. 1A is a graph depicting KRAS (also referred to as K-Ras) expression in human endometrial stromal cells (hESCs) from women with endometriosis carrying wild-type (WT) or the variant allele of KRAS gene at the LCS6 site. hESCs were obtained from endometrium of 10 women without endometriosis. Eutopic endomentrium was obtained from 10 women with endometriosis carrying WT KRAS and 10 women with endometriosis carry the variant allele of KRAS at the LCS6 site. q-RT-PCR results show comparatively low levels of KRAS mRNA in normal endometrium, increased KRAS mRNA in hESC from women with endometriosis and the WT KRAS allele (p=0.0007) and highest expression of KRAS mRNA in hESC carrying the variant KRAS allele (p=0.00018 when compared to normal hESC, p=0.0049 when compared to hESC from endometriosis patients with WT allele). *Difference is significant when compared to normal hESC. **Difference is significant when compared to hESC from women with endometriosis homozygous for the WT KRAS allele.

FIG. 1B is a photograph of a Western blot depicting KRAS expression in human endometrial stromal cells (hESCs) from women with endometriosis carrying wild-type (WT) or the variant allele of KRAS gene at the LCS6 site. hESCs were obtained from endometrium of 10 women without endometriosis. Eutopic endomentrium was obtained from 10 women with endometriosis carrying WT KRAS and 10 women with endometriosis carry the variant allele of KRAS at the LCS6 site. Western blot results show a 2.8-fold increase in KRAS protein in hESC with the variant allele.

FIG. 2 is a series of graphs depicting let-7 miRNA family expression in hESC from 10 subjects without endometriosis (normal controls), 10 subjects with endometriosis carrying WT KRAS and 10 subjects with endometriosis and the variant allele of KRAS gene. q-RT-PCR results show a trend towards decreased transcript levels of all let-7 miRNAs in hESC from subjects with endometriosis compared to normal hESC from subjects without endometriosis (normal control). Endometrial cells from subjects with endometriosis with the LCS6 variant in the KRAS gene showed lower levels of let-7a, let-7b and let-7e compared to hESC from endometriosis with non-variant KRAS. *p=0.0047 (let-7a), 0.003 (let-7b) and 0.05 (let-7c), respectively. Let-7a and let-7b were also lower in KRAS-variant cells from women with endometriosis compared to normal hESC (*p=0.05 (let-7a) and 0.02 (let-7b), respectively).

FIG. 3 is a graph depicting the effect of siRNA mimicking let-7 action on luciferase expression from the reporter plasmid carrying WT or variant KRAS allele in Dual Luciferase Reporter assay. Normal hESC were co-transfected with pGL3 vector carrying the variant LCS6 of the KRAS gene and either siRNA modified to bind the variant LCS6 or a negative control RNA sequence. Luciferase activity from the reporter plasmid carrying the KRAS-variant allele was greatly increased compared to that from a reporter plasmid containing non-variant allele at baseline. There was a 70% reduction in luciferase activity when the KRAS-variant allele was co-transfected with siRNA designed to bind the LCS6 site (p=0.045). No significant decrease in luciferase activity was obtained after co-transfecting this reporter plasmid with a control RNA sequence. The pGL3 control vector was used to assess transfection efficiency. Transfection with pGL3 carrying non-variant LCS6 of KRAS gene resulted in minimal luciferase activity likely due to inhibitory effects of endogenous let-7 miRNAs. Each experiment was carried out in duplicate in three separate experiments using cells from individual subjects. WT KRAS, hESC transfected with WT KRAS; KRAS V (KRAS variant), hESC transfected with KRAS variant allele; KRAS V+siRNA, hESC cotransfected with KRAS variant allele and siRNA; KRAS V+siRNA NC (negative control siRNA), hESC cotransfected with KRAS variant allele and siRNA negative control; pGL3 PC, hESC transfected with pGL3 control vector.

FIG. 4A is a graph depicting the effect of the KRAS variant allele on proliferation and invasion capacity of hESC. The effect of the KRAS variant allele on proliferation of hESC from women with endometriosis was determined by BrdU incorporation. There was a 71% increase (*p=0.04) in the BrdU label in hESC with the variant allele (n=5) versus WT KRAS LCS6 (n=5). These results indicated an increase in cell proliferation rate of hESC containing the mutant KRAS LCS6.

FIG. 4B is a graph depicting the effect of the KRAS variant allele on hESC invasion capacity. An invasion assay in which hESC from women with and without endometriosis and with the WT, non-variant, or, alternatively, the variant KRAS allele was used to determine the ability to invade extracellular matrix. There was a significant increase in the invasion of hESC containing the variant allele (n=9) compared to hESC without the variant allele (n=6) (*p=0.013). The difference between normal cells (n=6) and cells from endometriosis with WT KRAS was not significant.

FIG. 5A-C is a series of photographs depicting morphological and molecular features of endometriotic lesions containing the WT non-variant or variant alleles of the KRAS LCS6. Cultured endometrial stromal cells were injected under the kidney capsule of immunodeficient mice. A. Morphological appearance of the lesions under the kidney capsule of immune deficient mice 1 month after transplantation of hESC either with or without the variant KRAS. In all cases the transplanted endometrial cells formed endometriosis lesions with both glandular and stromal components. B. Proliferation marker expression in endometriotic lesions in mice. Nuclear staining for PCNA was more prominent in epithelium and stroma of the lesions formed by KRAS variant positive cells (54±5% and 56±6% in epithelium and stroma, respectively), compared to those derived from normal cells (8±4% and 34±6% in epithelium and stroma, respectively; p=0.02 and p 0.043, respectively) indicating higher proliferation levels in these cells. C. PR expression in endometriotic lesions with WT non-variant or variant KRAS allele in mice. Lesions created by hESC carrying KRAS variant allele were characterized by a smaller number of nuclei stained positively for PR in both glandular and stromal cells. The epithelium of the lesions with variant cells was found to have only 35±5% of nuclei positively stained compared to 75±3%, in the lesions with WT KRAS (p=0.02). Only 13±8% of nuclei of stromal cells in KRAS variant lesions were found to express PR compared to 78±7% in the non-variant lesions (p=0.028). Scale bar represents 25 mm.

### DETAILED DESCRIPTION

Endometriosis is a benign invasive estrogen dependent disorder characterized by the presence of endometrial glands and stroma outside the uterus. It is found in 10-15% of reproductive age women with more than 70 million affected worldwide (Endometriosis Research Center. Understanding endometriosis: past, present and future. The National Women's Health Information Council 2005; Bulun, S E. N Engl J Med 2009; 360:268-79; Hemmings, R, et al. Fertil Steril 2004; 81:1513-21; Gao, X, et al. Fertil Steril. 2006; 86:1561-72). Endometriosis has a dramatic effect on health and quality of life; it most commonly presents with chronic pelvic pain and causes infertility in up to 50% of women with the disease (Fourquet J, et al. (2010) Fertil Steril 93: 2424-2428). The yearly direct medical costs and indirect economic impact totals more than $22 billion in the United States alone (Practice Committee of the American Society for Reproductive Medicine. Endometriosis and Infertility. Fertil Steril 2006; 86 Suppl 4:156-60; Simoens, S, et al. Hum Reprod Update 2007; 13:395-404).

Although multiple medical therapies including oral contraceptives, danazol, progestins, GnRH analogues and aromatase inhibitors are commonly prescribed, there is no established cure for endometriosis; all treatments suppress the growth of both endometriosis and normal endometrium through hormonal mechanisms but none target disease specific pathways. Surgical intervention has proven to be an effective treatment; however the estimated recurrence rate still remains over 20% at 2 years and 50% at 5 years after laparoscopic surgery (Hadfleld, R et al. Hum Reprod 1996; 11:878-80). In the United States the mean delay in diagnosis is approximately 11 years (Guo, S-W. Hum Reprod Update 2009; 15:441-61). There is an obvious need to understand the biological basis of the disease in order to devise specific treatments, allow early diagnosis and potentially provide a means of prevention.

While the etiology and pathogenesis of endometriosis remain an active area of investigation, there is a genetic predisposition with a seven fold risk of endometriosis in women whose mother or sister has the disease (Simpson, J L, et al. Am J Obstet Gynecol. 1980; 137:327-31; Moen, M H, Magnus, P. Acta Obstet Gynecol Scand. 1993; 72(7):560-4; Simpson, J L, Bischoff, F Z. Ann NY Acad Sci. 2002; 955:239-51; Bischoff, F Z, Simpson, J L. Hum Reprod Update. 2000; 6(1):37-44). It has been previously shown both in human studies a well as murine models, that some genes are expressed differentially in eutopic endometrium of endometriosis patients compared to normal endometrium (Taylor, H S et al. Hum Reprod. 1999; 14(5):1328-31; Kao, L C, et al. Endocrinology. 2003; 144(7):2870-81; Lee, B, et al. Biol Reprod. 2009; 80(1):79-85). However no specific gene responsible for the disease has been identified so far in humans (Moen, M H, Magnus, P. Acta Obstet Gynecol Scand. 1993; 72(7):560-4; Simpson, J L, Bischoff, F Z. Ann NY Acad Sci. 2002; 955:239-51; Bischoff, F Z, Simpson, J L. Hum Reprod Update. 2000; 6(1):37-44). Two large genome wide association studies (GWAS) have linked disease susceptibility to 7p15.2 (a region between NFE2L3 and HOXA10) and to 9p21 in the CDKN2BAS gene with odds ratios of 1.22 and 1.44, respectively (Painter, J N, et al. Nat Gen 2011; 43:51-54; Uno, S, et al. Nature Gen 2010; 42, 707-711). These loci identify genetic linkage but have not been demonstrated to be involved in the pathophysiology of endometriosis.

No human studies have linked or identified alterations in the gene responsible for the only known murine model of spontaneous endometriosis. Activation of oncogenic KRAS gene through Cre-mediated transformation in the ovarian surface epithelium results in the de novo formation of the lesion with endometriotic morphology (Dinulescu D M, et al. (2005) Nat Med 11: 63-70). Moreover, a recent study demonstrated that activation of mutated KRAS in transplanted endometrium in mice triggered endometriosis formation and long-term survival of the lesions (Cheng C W, et al. (2011) J Pathol 224: 261-2269). However, despite thorough mutational analyses of KRAS in human endometriosis by several groups, no activating mutations in the coding regions of this gene have been found (Amemiya S, et al. (2004) Int J Gynecol Obstet 86: 371-376; Otsuka J, et al. (2004) Med Electron Microsc 37: 188-192; Vercellini P, et al. (1994) Gynecol Obstet Invest 38: 70-71; Zhao Z Z, et al. (2006) Mol Hum Reprod 12: 671-676).

Other possible mechanisms can alter the regulation of KRAS gene expression may be involved in the pathogenesis of human endometriosis. MicroRNAs (miRNAs) are small (20-22 nucleotides long) non-coding RNAs that degrade or prevent translation of their target genes by binding to the highly evolutionarily conserved 3′ untranslated regions (UTRs) of mRNAs (Carletti, M Z, Christenson, L K. J Anim Sci 2009; 87:29-38; Esquela-Kerscher, A, Slack, F J. Nat Rev Can 2006; 6:259-269; Calin, G A, et al. Proc Natl Acad Sci USA 2004; 101: 2999-3004). Single nucleotide polymorphisms within miRNAs or miRNA binding sites can alter gene expression and result in various pathological processes including malignant transformation (Esquela-Kerscher, A, Slack, F J. Nat Rev Can 2006; 6:259-269; Calin, G A, et al. Proc Natl Acad Sci USA 2004; 101: 2999-3004; Yang, H, et al. Cancer Res. 2008; 68(7):2530-7; Croce, C M. Nat Rev Gen 2009; 10:704-714). KRAS is known to be regulated in a microRNA dependent manner (Johnson, S M, et al. Cell. 2005; 120:635-647).

Lethal-7 (let-7), a founding member of the miRNA family in C. elegans, plays an important role in determining cell fate (Reinhart, B, et al. Nature. 2000; 403:901-906). Human orthologs include let-7a, let-7b, let-7c, let-7d, let-7e, let-7f, let-7g, and let-7i that inhibit cell growth and act as tumor suppressors (Reinhart, B, et al. Nature. 2000; 403:901-906; Roush, S, Slack, F J. Trends in Cell Biol. 2008; 18:505-516). Abnormal levels of these miRNAs are found in several human cancers (Takamizawa, J, et al. Cancer Res. 2004; 64:3753-3756; Inamura, K, et al. Lung Cancer. 2007; 58: 392-396; Jakymiw, A, et al. Genes, chromosomes cancer. 2010; 49:549-559; Jerome, T, et al. Current Genomics. 2007; 8: 229-233).

KRAS is a crucial target of let-7 miRNAs (Johnson, S M, et al. Cell. 2005; 120:635-647). KRAS expression is down regulated through 10 let-7 complementary sites (LCS) found in the 3′UTR of the KRAS gene (Chin, L J, et al. Cancer Res. 2008; 68:8535-8540). One of these LCSs (LCS6) is known to harbor a single nucleotide polymorphism (SNP) (T→G in the fourth position) which modifies the let-7 binding capacity of KRAS in lung cancer cells (Chin, L J, et al. Cancer Res. 2008; 68:8535-8540). The incidence of this SNP in the general population is 5.8% (Chin, L J, et al. Cancer Res. 2008; 68:8535-8540). This SNP is thought to lead to increased levels of KRAS protein thus resulting in alternative activation of Ras signaling and tumorigenesis. This variant allele is associated with an increased risk of the development of non-small cell lung cancer in people with only a moderate smoking history and is also a marker of poor prognosis in oral cancer (Chin, L J, et al. Cancer Res. 2008; 68:8535-8540; Christensen, B C, et al. Carcinogenesis. 2009; 30(6):1003-7). Similarly, this SNP has been identified in more than 25% of patients with ovarian cancer and is a marker of an increased risk of developing epithelial ovarian cancer (Ratner, E, et al. Cancer Res. 2010; 70:6509-15). This SNP has also been associated with an increased risk of triple negative breast cancer and unique tumor gene expression (Paranj ape et al., Lancet oncology).

The Ras pathway is activated by the presence of this previously identified SNP in LCS6 in the 3′UTR of KRAS in patients with endometriosis. The studies provided herein demonstrate the increased prevalence of this variant allele in women with endometriosis. The presence of this SNP results in elevated KRAS protein expression causing increased proliferation, migration and invasion of human endometrial stromal cells (hESCs).

### Endometriosis

Endometriosis occurs when endometrial tissue is found outside the uterus. The most widely accepted hypothesis for why endometriosis occurs is that endometrial cells are transported from the uterine cavity to ectopic sites where they become implanted by retrograde flow of menstrual tissue through the fallopian tubes. The lymphatic or circulatory system could transport endometrial cells to distant sites within the body.

The cells of ectopic endometrial growths consist of glands and stroma that are identical to intrauterine endometrium. Consequently, these tissues contain hormone receptors such as estrogen and progesterone receptors, and respond to changes in hormone levels in the body.

The tissue develops into growths that respond to hormonal changes resulting from the menstral cycle. Accordingly, just as endometrial tissue would do in the uterus, the tissue within ectopic endometrial growths builds up, breaks down, and sheds with each menstrual cycle. Also in accordance with the role of endometrial tissue in the uterus, the tissue that comprises an ectopic endometrial growth collects and sheds blood. The result of ectopic blood shedding is a number of signs or symptoms of endometriosis, including, but not limited to, internal bleeding, breakdown of the tissue of the growth, inflammation, infection, pain, scarring and adhesions, infertility (due to a combination of one or more of the preceding factors, including distortion of the pelvic architecture or hormone regime that may interfere with the ovarian cycle of egg release, form scar tissue surrounding the ovary decreasing the amount of surface area available for egg release, or interfere with the ability of the fallopian tubes to pick up an egg released by the ovary and/or transport that egg). Moreover, the bleeding, tissue damage, and inflammation events associated with endometriosis cause the peritoneal fluid to contain an increased number of immunological scavenger cells (i.e., primary leukocytes, white blood cells, microphages, macrophages, etc.), which may destroy sperm cells and contribute to infertility.

The term “symptom” is meant to describe an indication of disease, illness, injury, or that something is not right in the body. Symptoms are felt or noticed by the individual experiencing the symptom, but may not easily be noticed by others. Others are defined as non-health-care professionals.

The term “sign” is also meant to describe an indication that something is not right in the body. In contrast to symptoms, signs can be seen by a doctor, nurse, or other health care professional. For instance, a symptom is something a patient can report to a friend or a physician, whereas a sign may be an indication that a physician notices during a medical exam for following a medical test.

Other common signs or symptoms include recurring pelvic pain which often coincides with mild to severe cramping occurring on one or both sides of the pelvis, the lower back, the rectal area, and down the legs. Moreover, a sign or symptom of endometriosis includes, but is not limited to dysmenorrhea, dyspareunia, and dysuria. Dysmenorrhea is characterized by severe uterine and/or lower back pain (sharp, throbbing, dull, nauseating, burning, or shooting pain) during menstruation which may progressively worsen over time. Dysmenorrhea may coexist with excessively heavy blood loss, known as menorrhagia. Dyspareunia is characterized as painful sexual intercourse, which in the context of endometriosis, is likely caused by the presence of one or more ectopic endometrial growths on the reproductive organs and within the abdomen. Dysuria is characterized by painful urination, often accompanied by increased urinary urgency and frequency. Similarly dyschesia is by painful defecation. Leg pain, including throbbing sensations, is a common sign or symptom of endometriosis. More severe forms of endometriosis are accompanied by shooting pains and pressure-type within the pelvis and rectum.

Symptoms can vary depending on location of the ectopic endometrial growths. If a growth is located within the large intestine, the most common sign or symptoms include, but are not limited to pin during defecation, abdominal bloating, rectal bleeding during menses, and/or a combination thereof. Signs or symptoms of an ectopic endometrial growth occurring in the bladder include, but are not limited to, dysuria, hematuria, suprapubic pain, and/or a combination thereof. Most commonly, endometrial tissue is found on one or both of the ovaries. Signs or symptoms of an ectopic endometrial growth occurring one or both of the ovaries include, but are not limited to, formation of an endometrioma that may rupture or leak and cause acute abdominal pain and peritoneal signs. An endometrioma is a 2- to 10-cm cystic mass localized to an ovary. Signs or symptoms of an ectopic endometrial growth occurring in adnexal structures of the uterus (e.g. the ovaries, fallopian tubes, and structures of the broad ligament) include, but are not limited to, formation of adnexal adhesions, and consequently, formation of a pelvic mass. Signs or symptoms of an ectopic endometrial growth occurring in extrapelvic structures include, but are not limited to, delocalized abdominal pain.

The severity of a sign or symptom of endometriosis presented by an individual may not correlate directly with the stage or severity of endometriosis, as diagnosed surgically, within that individual.

Commonly, endometrial growths are found in the abdomen, on the ovaries, fallopian tubes, and ligaments that support the uterus; the area between the vagina and rectum; the outer surface of the uterus; and the lining of the pelvic cavity. Alternatively, or in addition, endometrial growths may include the bladder, bowel, vagina, cervix, vulva, and in abdominal surgical scars. Less commonly endometrial growths are found in other locations, including the upper and lower limbs.

Pelvic examinations of an individual with endometriosis may be normal. Alternatively, a physician or diagnostician may discover a retroverted and fixed uterus, enlarged ovaries, fixed ovarian masses, thickened rectovaginal septum, induration of the cul-de-sac, and/or nodules on the uterosacral ligament. Ectopic endothelial growths are rarely found on or within the vulva, cervix, vagina, umbilicus, or surgical scars.

Prior to development of the methods described herein, diagnosis of endometriosis was made only by laparoscopy. Laproscopy is a surgical procedure that is performed under anesthesia. The results of this procedure indicate the location, size, and extent of ectopic endometrial growths.

The American Society for Reproductive Medicine (ASRM) classifies endometriosis as stage I (minimal), II (mild), III (moderate), or IV (severe) (The American Society for Reproductive Medicine. Feral Steril 1997; 67: 817-21; the contents of which are incorporated herein in their entirety). This classification is based upon the number, location, and depth of ectopic endometrial growths as well as the presence and character of adhesions. Table 1 provides a simplified and condensed version of the ASRM classification.

TABLE 1
Diagnostic Stages of Endometriosis*

| Stage | Progression | Tissue Description |
|---|---|---|
| I | Minimal | Presentation of 2-3 superficial |
| | | implants. |
| II | Mild | Appearance of more implants that |
| | | occur within deeper layers of tissue. |
| III | Moderate | Many deep implants in combination |
| | | with minor/small endometriomas on |
| | | one or both ovaries. May also present |
| | | filmy adhesions. |
| IV | Severe | Persistance of deep implants, |
| | | enlargement of endometriomas on |
| | | one or both ovaries, development of |
| | | dense adhesions. |

*For more information, see Merck Manual, which is publicly available at www.merck.com/mmpe/sec18/ch247/ch247a.html.

Endometriosis is treated by a variety of methods including pain management, hormonal therapy, surgery, and alternative medicine. Pain management for this chronic condition ranges from over-the-counter to prescription-strength drugs. Hormonal therapies attempt to attenuate ovulation by use of oral contraceptives (e.g. progestin, the combination of estrogen and progestin,), progesterone and progestins, testosterone derivatives (e.g. danazol), and gonadotropin releasing hormone (GnRH) agonists or antagonists (e.g. leuprolide (Lupron, Eligard), buserelin (Suprefact, Suprecor), nafarelin (Synarel), histrelin (Supprelin), goserelin (Zoladex), deslorelin (Suprelorin, Ovuplant) and aromatase inhibitors (Femara). Finally, laproscopy or laparotomy may be performed to remove the ectopic endometrial growths. In the most severe situations, major surgery is performed (e.g. hysterectomy, removal of all growths, or removal of ovaries). Removal of these growths can provide pain relief or increase the odds of becoming pregnant. A combination of the any one or more of these treatments has been used to decrease symptoms.

Individuals who are at an increased risk of developing endometriosis include, but are not limited to, individuals who carry the KRAS variant, first-degree relatives of women with endometriosis, women who delay childbearing, women who have shortened menstrual cycles (e.g. a cycle of less than 27 days), women with menses that are abnormally long (a period lasting longer than 8 days), women who have müllerian duct anomalies, women who are infertile, women aged 15-44, women who have one or more of the preceding risk factors, and women aged 15-44 who have relatives with endometriosis.

Several factors are protective against the development of endometriosis, including, but not limited to, multiple pregnancies, use of low-dose oral contraceptives, and regular exercise. Thus, individuals who are at risk of developing endometriosis and who have none of the above protective factors are at particular risk for developing endometriosis. Moreover, to prevent endometriosis, these factors may be added to a therapeutic regime for those individuals who are identified as being at increased risk for developing endometriosis.

### KRAS Variant

The invention is based, in part, upon the unexpected discovery that the presence of a SNP in the 3′ untranslated region (UTR) of KRAS, referred to herein as the “KRAS variant,” is predictive of an individual's risk of developing endometriosis and an individual's response to treatment for endometriosis. The KRAS variant is located in LCS6, the wild type and variant sequence of which is provided below.

There are three human RAS genes comprising HRAS, KRAS, and NRAS. Each gene comprises multiple miRNA complementary sites in the 3′UTR of their mRNA transcripts. Specifically, each human RAS gene comprises multiple let-7 complementary sites (LCSs). The let-7 family-of-microRNAs (miRNAs) includes global genetic regulators important in controlling lung cancer oncogene expression by binding to the 3′UTRs (untranslated regions) of their target messenger RNAs (mRNAs).

Specifically, the term “let-7 complementary site” is meant to describe any region of a gene or gene transcript that binds a member of the let-7 family of miRNAs. Moreover, this term encompasses those sequences within a gene or gene transcript that are complementary to the sequence of a let-7 family miRNA. The term “complementary” describes a threshold of binding between two sequences wherein a majority of nucleotides in each sequence are capable of binding to a majority of nucleotides within the other sequence in trans.

The Human KRAS 3′ UTR comprises 8 LCSs named LCS1-LCS8, respectively. For the following sequences, thymine (T) may be substituted for uracil (U). LCS 1 comprises the sequence GACAGUGGAAGUUUUUUUUUCCUCG (SEQ ID NO: 1). LCS2 comprises the sequence AUUAGUGUCAUCUUGCCUC (SEQ ID NO: 2). LCS3 comprises the sequence AAUGCCCUACAUCUUAUUUUCCUCA (SEQ ID NO: 3). LCS4 comprises the sequence GGUUCAAGCGAUUCUCGUGCCUCG (SEQ ID NO: 4). LCS5 comprises the sequence GGCUGGUCCGAACUCCUGACCUCA (SEQ ID NO: 5). LCS6 comprises the sequence GAUUCACCCACCUUGGCCUCA (SEQ ID NO: 6). LCS7 comprises the sequence GGGUGUUAAGACUUGACACAGUACCUCG (SEQ ID NO: 7). LCS8 comprises the sequence AGUGCUUAUGAGGGGAUAUUUAGGCCUC (SEQ ID NO: 8).

Human KRAS has two wild type forms, encoded by transcripts a and b, which are provided below as SEQ ID NOs: 9 and 10, respectively. The sequences of each human KRAS transcript, containing the LCS6 SNP, are provided below as SEQ ID NOs: 11 and 12.

Human KRAS, transcript variant a, is encoded by the following mRNA sequence (NCBI Accession No. NM_{—}033360 and SEQ ID NO: 9) (untranslated regions are bolded, LCS6 is underlined):

| | |
|---|---|
| 1 | ggccgcggcg gcggaggcag cagcggcggc ggcagtggcg gcggcgaagg tggcggcggc |
| 61 | tcggccagta ctcccggccc ccgccatttc ggactgggag cgagcgcggc gcaggcactg |
| 121 | aaggcggcgg cggggccaga ggctcagcgg ctcccaggtg cgggagagag gcctgctgaa |
| 181 | aatgactgaa tataaacttg tggtagttgg agctggtggc gtaggcaaga gtgccttgac |
| 241 | gatacagcta attcagaatc attttgtgga cgaatatgat ccaacaatag aggattccta |
| 301 | caggaagcaa gtagtaattg atggagaaac ctgtctcttg gatattctcg acacagcagg |
| 361 | tcaagaggag tacagtgcaa tgagggacca gtacatgagg actggggagg gctttctttg |
| 421 | tgtatttgcc ataaataata ctaaatcatt tgaagatatt caccattata gagaacaaat |
| 481 | taaaagagtt aaggactctg aagatgtacc tatggtccta gtaggaaata aatgtgattt |
| 541 | gccttctaga acagtagaca caaaacaggc tcaggactta gcaagaagtt atggaattcc |
| 601 | ttttattgaa acatcagcaa agacaagaca gagagtggag gatgcttttt atacattggt |
| 661 | gagggagatc cgacaataca gattgaaaaa aatcagcaaa gaagaaaaga ctcctggctg |
| 721 | tgtgaaaatt aaaaaatgca ttataatgta atctgggtgt tgatgatgcc ttctatacat |
| 781 | tagttcgaga aattcgaaaa cataaagaaa agatgagcaa agatggtaaa aagaagaaaa |
| 841 | agaagtcaaa gacaaagtgt gtaattatgt aaatacaatt tgtacttttt tcttaaggca |
| 901 | tactagtaca agtggtaatt tttgtacatt acactaaatt attagcattt gttttagcat |
| 961 | tacctaattt ttttcctgct ccatgcagac tgttagcttt taccttaaat gcttatttta |
| 1021 | aaatgacagt ggaagttttt ttttcctcta agtgccagta ttcccagagt tttggttttt |
| 1081 | gaactagcaa tgcctgtgaa aaagaaactg aatacctaag atttctgtct tggggttttt |
| 1141 | ggtgcatgca gttgattact tcttattttt cttaccaatt gtgaatgttg gtgtgaaaca |
| 1201 | aattaatgaa gcttttgaat catccctatt ctgtgtttta tctagtcaca taaatggatt |
| 1261 | aattactaat ttcagttgag accttctaat tggtttttac tgaaacattg agggaacaca |
| 1321 | aatttatggg cttcctgatg atgattcttc taggcatcat gtcctatagt ttgtcatccc |
| 1381 | tgatgaatgt aaagttacac tgttcacaaa ggttttgtct cctttccact gctattagtc |
| 1441 | atggtcactc tccccaaaat attatatttt ttctataaaa agaaaaaaat ggaaaaaaat |
| 1501 | tacaaggcaa tggaaactat tataaggcca tttccttttc acattagata aattactata |
| 1561 | aagactccta atagcttttc ctgttaaggc agacccagta tgaaatgggg attattatag |
| 1621 | caaccatttt ggggctatat ttacatgcta ctaaattttt ataataattg aaaagatttt |
| 1681 | aacaagtata aaaaattctc ataggaatta aatgtagtct ccctgtgtca gactgctctt |
| 1741 | tcatagtata actttaaatc ttttcttcaa cttgagtctt tgaagatagt tttaattctg |
| 1801 | cttgtgacat taaaagatta tttgggccag ttatagctta ttaggtgttg aagagaccaa |
| 1861 | ggttgcaagg ccaggccctg tgtgaacctt tgagctttca tagagagttt cacagcatgg |
| 1921 | actgtgtccc cacggtcatc cagtgttgtc atgcattggt tagtcaaaat ggggagggac |
| 1981 | tagggcagtt tggatagctc aacaagatac aatctcactc tgtggtggtc ctgctgacaa |
| 2041 | atcaagagca ttgcttttgt ttcttaagaa aacaaactct tttttaaaaa ttacttttaa |
| 2101 | atattaactc aaaagttgag attttggggt ggtggtgtgc caagacatta attttttttt |
| 2161 | taaacaatga agtgaaaaag ttttacaatc tctaggtttg gctagttctc ttaacactgg |
| 2221 | ttaaattaac attgcataaa cacttttcaa gtctgatcca tatttaataa tgctttaaaa |
| 2281 | taaaaataaa aacaatcctt ttgataaatt taaaatgtta cttattttaa aataaatgaa |
| 2341 | gtgagatggc atggtgaggt gaaagtatca ctggactagg aagaaggtga cttaggttct |
| 2401 | agataggtgt cttttaggac tctgattttg aggacatcac ttactatcca tttcttcatg |
| 2461 | ttaaaagaag tcatctcaaa ctcttagttt ttttttttta caactatgta atttatattc |
| 2521 | catttacata aggatacact tatttgtcaa gctcagcaca atctgtaaat ttttaaccta |
| 2581 | tgttacacca tcttcagtgc cagtcttggg caaaattgtg caagaggtga agtttatatt |
| 2641 | tgaatatcca ttctcgtttt aggactcttc ttccatatta gtgtcatctt gcctccctac |
| 2701 | cttccacatg ccccatgact tgatgcagtt ttaatacttg taattcccct aaccataaga |
| 2761 | tttactgctg ctgtggatat ctccatgaag ttttcccact gagtcacatc agaaatgccc |
| 2821 | tacatcttat ttcctcaggg ctcaagagaa tctgacagat accataaagg gatttgacct |
| 2881 | aatcactaat tttcaggtgg tggctgatgc tttgaacatc tctttgctgc ccaatccatt |
| 2941 | agcgacagta ggatttttca aacctggtat gaatagacag aaccctatcc agtggaagga |
| 3001 | gaatttaata aagatagtgc tgaaagaatt ccttaggtaa tctataacta ggactactcc |
| 3061 | tggtaacagt aatacattcc attgttttag taaccagaaa tcttcatgca atgaaaaata |
| 3121 | ctttaattca tgaagcttac tttttttttt tggtgtcaga gtctcgctct tgtcacccag |
| 3181 | gctggaatgc agtggcgcca tctcagctca ctgcaacctc catctcccag gttcaagcga |
| 3241 | ttctcgtgcc tcggcctcct gagtagctgg gattacaggc gtgtgccact acactcaact |
| 3301 | aatttttgta tttttaggag agacggggtt tcaccctgtt ggccaggctg gtctcgaact |
| 3361 | cctgacctca agtgattcac ccaccttggc ctcataaacc tgttttgcag aactcattta |
| 3421 | ttcagcaaat atttattgag tgcctaccag atgccagtca ccgcacaagg cactgggtat |
| 3481 | atggtatccc caaacaagag acataatccc ggtccttagg tagtgctagt gtggtctgta |
| 3541 | atatcttact aaggcctttg gtatacgacc cagagataac acgatgcgta ttttagtttt |
| 3601 | gcaaagaagg ggtttggtct ctgtgccagc tctataattg ttttgctacg attccactga |
| 3661 | aactcttcga tcaagctact ttatgtaaat cacttcattg ttttaaagga ataaacttga |
| 3721 | ttatattgtt tttttatttg gcataactgt gattctttta ggacaattac tgtacacatt |
| 3781 | aaggtgtatg tcagatattc atattgaccc aaatgtgtaa tattccagtt ttctctgcat |
| 3841 | aagtaattaa aatatactta aaaattaata gttttatctg ggtacaaata aacaggtgcc |
| 3901 | tgaactagtt cacagacaag gaaacttcta tgtaaaaatc actatgattt ctgaattgct |
| 3961 | atgtgaaact acagatcttt ggaacactgt ttaggtaggg tgttaagact tacacagtac |
| 4021 | ctcgtttcta cacagagaaa gaaatggcca tacttcagga actgcagtgc ttatgagggg |
| 4081 | atatttaggc ctcttgaatt tttgatgtag atgggcattt ttttaaggta gtggttaatt |
| 4141 | acctttatgt gaactttgaa tggtttaaca aaagatttgt ttttgtagag attttaaagg |
| 4201 | gggagaattc tagaaataaa tgttacctaa ttattacagc cttaaagaca aaaatccttg |
| 4261 | ttgaagtttt tttaaaaaaa gctaaattac atagacttag gcattaacat gtttgtggaa |
| 4321 | gaatatagca gacgtatatt gtatcatttg agtgaatgtt cccaagtagg cattctaggc |
| 4381 | tctatttaac tgagtcacac tgcataggaa tttagaacct aacttttata ggttatcaaa |
| 4441 | actgttgtca ccattgcaca attttgtcct aatatataca tagaaacttt gtggggcatg |
| 4501 | ttaagttaca gtttgcacaa gttcatctca tttgtattcc attgattttt tttttcttct |
| 4561 | aaacattttt tcttcaaaca gtatataact ttttttaggg gatttttttt tagacagcaa |
| 4621 | aaactatctg aagatttcca tttgtcaaaa agtaatgatt tcttgataat tgtgtagtaa |
| 4681 | tgttttttag aacccagcag ttaccttaaa gctgaattta tatttagtaa cttctgtgtt |
| 4741 | aatactggat agcatgaatt ctgcattgag aaactgaata gctgtcataa aatgaaactt |
| 4801 | tctttctaaa gaaagatact cacatgagtt cttgaagaat agtcataact agattaagat |
| 4861 | ctgtgtttta gtttaatagt ttgaagtgcc tgtttgggat aatgataggt aatttagatg |
| 4921 | aatttagggg aaaaaaaagt tatctgcaga tatgttgagg gcccatctct ccccccacac |
| 4981 | ccccacagag ctaactgggt tacagtgttt tatccgaaag tttccaattc cactgtcttg |
| 5041 | tgttttcatg ttgaaaatac ttttgcattt ttcctttgag tgccaatttc ttactagtac |
| 5101 | tatttcttaa tgtaacatgt ttacctggaa tgtattttaa ctatttttgt atagtgtaaa |
| 5161 | ctgaaacatg cacattttgt acattgtgct ttcttttgtg ggacatatgc agtgtgatcc |
| 5221 | agttgttttc catcatttgg ttgcgctgac ctaggaatgt tggtcatatc aaacattaaa |
| 5281 | aatgaccact cttttaattg aaattaactt ttaaatgttt ataggagtat gtgctgtgaa |
| 5341 | gtgatctaaa atttgtaata tttttgtcat gaactgtact actcctaatt attgtaatgt |
| 5401 | aataaaaata gttacagtga caaaaaaaaa aaaaaa |

Human KRAS, transcript variant b, is encoded by the following mRNA sequence (NCBI Accession No. NM_{—}004985 and SEQ ID NO: 10) (untranslated regions are bolded, LCS6 is underlined):

| | |
|---|---|
| 1 | ggccgcggcg gcggaggcag cagcggcggc ggcagtggcg gcggcgaagg tggcggcggc |
| 61 | tcggccagta ctcccggccc ccgccatttc ggactgggag cgagcgcggc gcaggcactg |
| 121 | aaggcggcgg cggggccaga ggctcagcgg ctcccaggtg cgggagagag gcctgctgaa |
| 181 | aatgactgaa tataaacttg tggtagttgg agctggtggc gtaggcaaga gtgccttgac |
| 241 | gatacagcta attcagaatc attttgtgga cgaatatgat ccaacaatag aggattccta |
| 301 | caggaagcaa gtagtaattg atggagaaac ctgtctcttg gatattctcg acacagcagg |
| 361 | tcaagaggag tacagtgcaa tgagggacca gtacatgagg actggggagg gctttctttg |
| 421 | tgtatttgcc ataaataata ctaaatcatt tgaagatatt caccattata gagaacaaat |
| 481 | taaaagagtt aaggactctg aagatgtacc tatggtccta gtaggaaata aatgtgattt |
| 541 | gccttctaga acagtagaca caaaacaggc tcaggactta gcaagaagtt atggaattcc |
| 601 | ttttattgaa acatcagcaa agacaagaca gggtgttgat gatgccttct atacattagt |
| 661 | tcgagaaatt cgaaaacata aagaaaagat gagcaaagat ggtaaaaaga agaaaaagaa |
| 721 | gtcaaagaca aagtgtgtaa ttatgtaaat acaatttgta cttttttctt aaggcatact |
| 781 | agtacaagtg gtaatttttg tacattacac taaattatta gcatttgttt tagcattacc |
| 841 | taattttttt cctgctccat gcagactgtt agcttttacc ttaaatgctt attttaaaat |
| 901 | gacagtggaa gttttttttt cctctaagtg ccagtattcc cagagttttg gtttttgaac |
| 961 | tagcaatgcc tgtgaaaaag aaactgaata cctaagattt ctgtcttggg gtttttggtg |
| 1021 | catgcagttg attacttctt atttttctta ccaattgtga atgttggtgt gaaacaaatt |
| 1081 | aatgaagctt ttgaatcatc cctattctgt gttttatcta gtcacataaa tggattaatt |
| 1141 | actaatttca gttgagacct tctaattggt ttttactgaa acattgaggg aacacaaatt |
| 1201 | tatgggcttc ctgatgatga ttcttctagg catcatgtcc tatagtttgt catccctgat |
| 1261 | gaatgtaaag ttacactgtt cacaaaggtt ttgtctcctt tccactgcta ttagtcatgg |
| 1321 | tcactctccc caaaatatta tattttttct ataaaaagaa aaaaatggaa aaaaattaca |
| 1381 | aggcaatgga aactattata aggccatttc cttttcacat tagataaatt actataaaga |
| 1441 | ctcctaatag cttttcctgt taaggcagac ccagtatgaa atggggatta ttatagcaac |
| 1501 | cattttgggg ctatatttac atgctactaa atttttataa taattgaaaa gattttaaca |
| 1561 | agtataaaaa attctcatag gaattaaatg tagtctccct gtgtcagact gctctttcat |
| 1621 | agtataactt taaatctttt cttcaacttg agtctttgaa gatagtttta attctgcttg |
| 1681 | tgacattaaa agattatttg ggccagttat agcttattag gtgttgaaga gaccaaggtt |
| 1741 | gcaaggccag gccctgtgtg aacctttgag ctttcataga gagtttcaca gcatggactg |
| 1801 | tgtccccacg gtcatccagt gttgtcatgc attggttagt caaaatgggg agggactagg |
| 1861 | gcagtttgga tagctcaaca agatacaatc tcactctgtg gtggtcctgc tgacaaatca |
| 1921 | agagcattgc ttttgtttct taagaaaaca aactcttttt taaaaattac ttttaaatat |
| 1981 | taactcaaaa gttgagattt tggggtggtg gtgtgccaag acattaattt tttttttaaa |
| 2041 | caatgaagtg aaaaagtttt acaatctcta ggtttggcta gttctcttaa cactggttaa |
| 2101 | attaacattg cataaacact tttcaagtct gatccatatt taataatgct ttaaaataaa |
| 2161 | aataaaaaca atccttttga taaatttaaa atgttactta ttttaaaata aatgaagtga |
| 2221 | gatggcatgg tgaggtgaaa gtatcactgg actaggaaga aggtgactta ggttctagat |
| 2281 | aggtgtcttt taggactctg attttgagga catcacttac tatccatttc ttcatgttaa |
| 2341 | aagaagtcat ctcaaactct tagttttttt tttttacaac tatgtaattt atattccatt |
| 2401 | tacataagga tacacttatt tgtcaagctc agcacaatct gtaaattttt aacctatgtt |
| 2461 | acaccatctt cagtgccagt cttgggcaaa attgtgcaag aggtgaagtt tatatttgaa |
| 2521 | tatccattct cgttttagga ctcttcttcc atattagtgt catcttgcct ccctaccttc |
| 2581 | cacatgcccc atgacttgat gcagttttaa tacttgtaat tcccctaacc ataagattta |
| 2641 | ctgctgctgt ggatatctcc atgaagtttt cccactgagt cacatcagaa atgccctaca |
| 2701 | tcttatttcc tcagggctca agagaatctg acagatacca taaagggatt tgacctaatc |
| 2761 | actaattttc aggtggtggc tgatgctttg aacatctctt tgctgcccaa tccattagcg |
| 2821 | acagtaggat ttttcaaacc tggtatgaat agacagaacc ctatccagtg gaaggagaat |
| 2881 | ttaataaaga tagtgctgaa agaattcctt aggtaatcta taactaggac tactcctggt |
| 2941 | aacagtaata cattccattg ttttagtaac cagaaatctt catgcaatga aaaatacttt |
| 3001 | aattcatgaa gcttactttt tttttttggt gtcagagtct cgctcttgtc acccaggctg |
| 3061 | gaatgcagtg gcgccatctc agctcactgc aacctccatc tcccaggttc aagcgattct |
| 3121 | cgtgcctcgg cctcctgagt agctgggatt acaggcgtgt gccactacac tcaactaatt |
| 3181 | tttgtatttt taggagagac ggggtttcac cctgttggcc aggctggtct cgaactcctg |
| 3241 | acctcaagtg attcacccac cttggcctca taaacctgtt ttgcagaact catttattca |
| 3301 | gcaaatattt attgagtgcc taccagatgc cagtcaccgc acaaggcact gggtatatgg |
| 3361 | tatccccaaa caagagacat aatcccggtc cttaggtagt gctagtgtgg tctgtaatat |
| 3421 | cttactaagg cctttggtat acgacccaga gataacacga tgcgtatttt agttttgcaa |
| 3481 | agaaggggtt tggtctctgt gccagctcta taattgtttt gctacgattc cactgaaact |
| 3541 | cttcgatcaa gctactttat gtaaatcact tcattgtttt aaaggaataa acttgattat |
| 3601 | attgtttttt tatttggcat aactgtgatt cttttaggac aattactgta cacattaagg |
| 3661 | tgtatgtcag atattcatat tgacccaaat gtgtaatatt ccagttttct ctgcataagt |
| 3721 | aattaaaata tacttaaaaa ttaatagttt tatctgggta caaataaaca ggtgcctgaa |
| 3781 | ctagttcaca gacaaggaaa cttctatgta aaaatcacta tgatttctga attgctatgt |
| 3841 | gaaactacag atctttggaa cactgtttag gtagggtgtt aagacttaca cagtacctcg |
| 3901 | tttctacaca gagaaagaaa tggccatact tcaggaactg cagtgcttat gaggggatat |
| 3961 | ttaggcctct tgaatttttg atgtagatgg gcattttttt aaggtagtgg ttaattacct |
| 4021 | ttatgtgaac tttgaatggt ttaacaaaag atttgttttt gtagagattt taaaggggga |
| 4081 | gaattctaga aataaatgtt acctaattat tacagcctta aagacaaaaa tccttgttga |
| 4141 | agttttttta aaaaaagcta aattacatag acttaggcat taacatgttt gtggaagaat |
| 4201 | atagcagacg tatattgtat catttgagtg aatgttccca agtaggcatt ctaggctcta |
| 4261 | tttaactgag tcacactgca taggaattta gaacctaact tttataggtt atcaaaactg |
| 4321 | ttgtcaccat tgcacaattt tgtcctaata tatacataga aactttgtgg ggcatgttaa |
| 4381 | gttacagttt gcacaagttc atctcatttg tattccattg attttttttt tcttctaaac |
| 4441 | attttttctt caaacagtat ataacttttt ttaggggatt tttttttaga cagcaaaaac |
| 4501 | tatctgaaga tttccatttg tcaaaaagta atgatttctt gataattgtg tagtaatgtt |
| 4561 | ttttagaacc cagcagttac cttaaagctg aatttatatt tagtaacttc tgtgttaata |
| 4621 | ctggatagca tgaattctgc attgagaaac tgaatagctg tcataaaatg aaactttctt |
| 4681 | tctaaagaaa gatactcaca tgagttcttg aagaatagtc ataactagat taagatctgt |
| 4741 | gttttagttt aatagtttga agtgcctgtt tgggataatg ataggtaatt tagatgaatt |
| 4801 | taggggaaaa aaaagttatc tgcagatatg ttgagggccc atctctcccc ccacaccccc |
| 4861 | acagagctaa ctgggttaca gtgttttatc cgaaagtttc caattccact gtcttgtgtt |
| 4921 | ttcatgttga aaatactttt gcatttttcc tttgagtgcc aatttcttac tagtactatt |
| 4981 | tcttaatgta acatgtttac ctggaatgta ttttaactat ttttgtatag tgtaaactga |
| 5041 | aacatgcaca ttttgtacat tgtgctttct tttgtgggac atatgcagtg tgatccagtt |
| 5101 | gttttccatc atttggttgc gctgacctag gaatgttggt catatcaaac attaaaaatg |
| 5161 | accactcttt taattgaaat taacttttaa atgtttatag gagtatgtgc tgtgaagtga |
| 5221 | tctaaaattt gtaatatttt tgtcatgaac tgtactactc ctaattattg taatgtaata |
| 5281 | aaaatagtta cagtgacaaa aaaaaaaaaa aa |

Human KRAS, transcript variant a, comprising the LCS6 SNP, is encoded by the following mRNA sequence (SEQ ID NO: 11) (untranslated regions are bolded, LCS6 is underlined, SNP is capitalized):

| | |
|---|---|
| 1 | ggccgcggcg gcggaggcag cagcggcggc ggcagtggcg gcggcgaagg tggcggcggc |
| 61 | tcggccagta ctcccggccc ccgccatttc ggactgggag cgagcgcggc gcaggcactg |
| 121 | aaggcggcgg cggggccaga ggctcagcgg ctcccaggtg cgggagagag gcctgctgaa |
| 181 | aatgactgaa tataaacttg tggtagttgg agctggtggc gtaggcaaga gtgccttgac |
| 241 | gatacagcta attcagaatc attttgtgga cgaatatgat ccaacaatag aggattccta |
| 301 | caggaagcaa gtagtaattg atggagaaac ctgtctcttg gatattctcg acacagcagg |
| 361 | tcaagaggag tacagtgcaa tgagggacca gtacatgagg actggggagg gctttctttg |
| 421 | tgtatttgcc ataaataata ctaaatcatt tgaagatatt caccattata gagaacaaat |
| 481 | taaaagagtt aaggactctg aagatgtacc tatggtccta gtaggaaata aatgtgattt |
| 541 | gccttctaga acagtagaca caaaacaggc tcaggactta gcaagaagtt atggaattcc |
| 601 | ttttattgaa acatcagcaa agacaagaca gagagtggag gatgcttttt atacattggt |
| 661 | gagggagatc cgacaataca gattgaaaaa aatcagcaaa gaagaaaaga ctcctggctg |
| 721 | tgtgaaaatt aaaaaatgca ttataatgta atctgggtgt tgatgatgcc ttctatacat |
| 781 | tagttcgaga aattcgaaaa cataaagaaa agatgagcaa agatggtaaa aagaagaaaa |
| 841 | agaagtcaaa gacaaagtgt gtaattatgt aaatacaatt tgtacttttt tcttaaggca |
| 901 | tactagtaca agtggtaatt tttgtacatt acactaaatt attagcattt gttttagcat |
| 961 | tacctaattt ttttcctgct ccatgcagac tgttagcttt taccttaaat gcttatttta |
| 1021 | aaatgacagt ggaagttttt ttttcctcta agtgccagta ttcccagagt tttggttttt |
| 1081 | gaactagcaa tgcctgtgaa aaagaaactg aatacctaag atttctgtct tggggttttt |
| 1141 | ggtgcatgca gttgattact tcttattttt cttaccaatt gtgaatgttg gtgtgaaaca |
| 1201 | aattaatgaa gcttttgaat catccctatt ctgtgtttta tctagtcaca taaatggatt |
| 1261 | aattactaat ttcagttgag accttctaat tggtttttac tgaaacattg agggaacaca |
| 1321 | aatttatggg cttcctgatg atgattcttc taggcatcat gtcctatagt ttgtcatccc |
| 1381 | tgatgaatgt aaagttacac tgttcacaaa ggttttgtct cctttccact gctattagtc |
| 1441 | atggtcactc tccccaaaat attatatttt ttctataaaa agaaaaaaat ggaaaaaaat |
| 1501 | tacaaggcaa tggaaactat tataaggcca tttccttttc acattagata aattactata |
| 1561 | aagactccta atagcttttc ctgttaaggc agacccagta tgaaatgggg attattatag |
| 1621 | caaccatttt ggggctatat ttacatgcta ctaaattttt ataataattg aaaagatttt |
| 1681 | aacaagtata aaaaattctc ataggaatta aatgtagtct ccctgtgtca gactgctctt |
| 1741 | tcatagtata actttaaatc ttttcttcaa cttgagtctt tgaagatagt tttaattctg |
| 1801 | cttgtgacat taaaagatta tttgggccag ttatagctta ttaggtgttg aagagaccaa |
| 1861 | ggttgcaagg ccaggccctg tgtgaacctt tgagctttca tagagagttt cacagcatgg |
| 1921 | actgtgtccc cacggtcatc cagtgttgtc atgcattggt tagtcaaaat ggggagggac |
| 1981 | tagggcagtt tggatagctc aacaagatac aatctcactc tgtggtggtc ctgctgacaa |
| 2041 | atcaagagca ttgcttttgt ttcttaagaa aacaaactct tttttaaaaa ttacttttaa |
| 2101 | atattaactc aaaagttgag attttggggt ggtggtgtgc caagacatta attttttttt |
| 2161 | taaacaatga agtgaaaaag ttttacaatc tctaggtttg gctagttctc ttaacactgg |
| 2221 | ttaaattaac attgcataaa cacttttcaa gtctgatcca tatttaataa tgctttaaaa |
| 2281 | taaaaataaa aacaatcctt ttgataaatt taaaatgtta cttattttaa aataaatgaa |
| 2341 | gtgagatggc atggtgaggt gaaagtatca ctggactagg aagaaggtga cttaggttct |
| 2401 | agataggtgt cttttaggac tctgattttg aggacatcac ttactatcca tttcttcatg |
| 2461 | ttaaaagaag tcatctcaaa ctcttagttt ttttttttta caactatgta atttatattc |
| 2521 | catttacata aggatacact tatttgtcaa gctcagcaca atctgtaaat ttttaaccta |
| 2581 | tgttacacca tcttcagtgc cagtcttggg caaaattgtg caagaggtga agtttatatt |
| 2641 | tgaatatcca ttctcgtttt aggactcttc ttccatatta gtgtcatctt gcctccctac |
| 2701 | cttccacatg ccccatgact tgatgcagtt ttaatacttg taattcccct aaccataaga |
| 2761 | tttactgctg ctgtggatat ctccatgaag ttttcccact gagtcacatc agaaatgccc |
| 2821 | tacatcttat ttcctcaggg ctcaagagaa tctgacagat accataaagg gatttgacct |
| 2881 | aatcactaat tttcaggtgg tggctgatgc tttgaacatc tctttgctgc ccaatccatt |
| 2941 | agcgacagta ggatttttca aacctggtat gaatagacag aaccctatcc agtggaagga |
| 3001 | gaatttaata aagatagtgc tgaaagaatt ccttaggtaa tctataacta ggactactcc |
| 3061 | tggtaacagt aatacattcc attgttttag taaccagaaa tcttcatgca atgaaaaata |
| 3121 | ctttaattca tgaagcttac tttttttttt tggtgtcaga gtctcgctct tgtcacccag |
| 3181 | gctggaatgc agtggcgcca tctcagctca ctgcaacctc catctcccag gttcaagcga |
| 3241 | ttctcgtgcc tcggcctcct gagtagctgg gattacaggc gtgtgccact acactcaact |
| 3301 | aatttttgta tttttaggag agacggggtt tcaccctgtt ggccaggctg gtctcgaact |
| 3361 | cctgacctca agtgatGcac ccaccttggc ctcataaacc tgttttgcag aactcattta |
| 3421 | ttcagcaaat atttattgag tgcctaccag atgccagtca ccgcacaagg cactgggtat |
| 3481 | atggtatccc caaacaagag acataatccc ggtccttagg tagtgctagt gtggtctgta |
| 3541 | atatcttact aaggcctttg gtatacgacc cagagataac acgatgcgta ttttagtttt |
| 3601 | gcaaagaagg ggtttggtct ctgtgccagc tctataattg ttttgctacg attccactga |
| 3661 | aactcttcga tcaagctact ttatgtaaat cacttcattg ttttaaagga ataaacttga |
| 3721 | ttatattgtt tttttatttg gcataactgt gattctttta ggacaattac tgtacacatt |
| 3781 | aaggtgtatg tcagatattc atattgaccc aaatgtgtaa tattccagtt ttctctgcat |
| 3841 | aagtaattaa aatatactta aaaattaata gttttatctg ggtacaaata aacaggtgcc |
| 3901 | tgaactagtt cacagacaag gaaacttcta tgtaaaaatc actatgattt ctgaattgct |
| 3961 | atgtgaaact acagatcttt ggaacactgt ttaggtaggg tgttaagact tacacagtac |
| 4021 | ctcgtttcta cacagagaaa gaaatggcca tacttcagga actgcagtgc ttatgagggg |
| 4081 | atatttaggc ctcttgaatt tttgatgtag atgggcattt ttttaaggta gtggttaatt |
| 4141 | acctttatgt gaactttgaa tggtttaaca aaagatttgt ttttgtagag attttaaagg |
| 4201 | gggagaattc tagaaataaa tgttacctaa ttattacagc cttaaagaca aaaatccttg |
| 4261 | ttgaagtttt tttaaaaaaa gctaaattac atagacttag gcattaacat gtttgtggaa |
| 4321 | gaatatagca gacgtatatt gtatcatttg agtgaatgtt cccaagtagg cattctaggc |
| 4381 | tctatttaac tgagtcacac tgcataggaa tttagaacct aacttttata ggttatcaaa |
| 4441 | actgttgtca ccattgcaca attttgtcct aatatataca tagaaacttt gtggggcatg |
| 4501 | ttaagttaca gtttgcacaa gttcatctca tttgtattcc attgattttt tttttcttct |
| 4561 | aaacattttt tcttcaaaca gtatataact ttttttaggg gatttttttt tagacagcaa |
| 4621 | aaactatctg aagatttcca tttgtcaaaa agtaatgatt tcttgataat tgtgtagtaa |
| 4681 | tgttttttag aacccagcag ttaccttaaa gctgaattta tatttagtaa cttctgtgtt |
| 4741 | aatactggat agcatgaatt ctgcattgag aaactgaata gctgtcataa aatgaaactt |
| 4801 | tctttctaaa gaaagatact cacatgagtt cttgaagaat agtcataact agattaagat |
| 4861 | ctgtgtttta gtttaatagt ttgaagtgcc tgtttgggat aatgataggt aatttagatg |
| 4921 | aatttagggg aaaaaaaagt tatctgcaga tatgttgagg gcccatctct ccccccacac |
| 4981 | ccccacagag ctaactgggt tacagtgttt tatccgaaag tttccaattc cactgtcttg |
| 5041 | tgttttcatg ttgaaaatac ttttgcattt ttcctttgag tgccaatttc ttactagtac |
| 5101 | tatttcttaa tgtaacatgt ttacctggaa tgtattttaa ctatttttgt atagtgtaaa |
| 5161 | ctgaaacatg cacattttgt acattgtgct ttcttttgtg ggacatatgc agtgtgatcc |
| 5221 | agttgttttc catcatttgg ttgcgctgac ctaggaatgt tggtcatatc aaacattaaa |
| 5281 | aatgaccact cttttaattg aaattaactt ttaaatgttt ataggagtat gtgctgtgaa |
| 5341 | gtgatctaaa atttgtaata tttttgtcat gaactgtact actcctaatt attgtaatgt |
| 5401 | aataaaaata gttacagtga caaaaaaaaa aaaaaa |

Human KRAS, transcript variant b, comprising the LCS6 SNP, is encoded by the following mRNA sequence (SEQ ID NO: 12) (untranslated regions are bolded, LCS6 is underlined, SNP is capitalized):

| | |
|---|---|
| 1 | ggccgcggcg gcggaggcag cagcggcggc ggcagtggcg gcggcgaagg tggcggcggc |
| 61 | tcggccagta ctcccggccc ccgccatttc ggactgggag cgagcgcggc gcaggcactg |
| 121 | aaggcggcgg cggggccaga ggctcagcgg ctcccaggtg cgggagagag gcctgctgaa |
| 181 | aatgactgaa tataaacttg tggtagttgg agctggtggc gtaggcaaga gtgccttgac |
| 241 | gatacagcta attcagaatc attttgtgga cgaatatgat ccaacaatag aggattccta |
| 301 | caggaagcaa gtagtaattg atggagaaac ctgtctcttg gatattctcg acacagcagg |
| 361 | tcaagaggag tacagtgcaa tgagggacca gtacatgagg actggggagg gctttctttg |
| 421 | tgtatttgcc ataaataata ctaaatcatt tgaagatatt caccattata gagaacaaat |
| 481 | taaaagagtt aaggactctg aagatgtacc tatggtccta gtaggaaata aatgtgattt |
| 541 | gccttctaga acagtagaca caaaacaggc tcaggactta gcaagaagtt atggaattcc |
| 601 | ttttattgaa acatcagcaa agacaagaca gggtgttgat gatgccttct atacattagt |
| 661 | tcgagaaatt cgaaaacata aagaaaagat gagcaaagat ggtaaaaaga agaaaaagaa |
| 721 | gtcaaagaca aagtgtgtaa ttatgtaaat acaatttgta cttttttctt aaggcatact |
| 781 | agtacaagtg gtaatttttg tacattacac taaattatta gcatttgttt tagcattacc |
| 841 | taattttttt cctgctccat gcagactgtt agcttttacc ttaaatgctt attttaaaat |
| 901 | gacagtggaa gttttttttt cctctaagtg ccagtattcc cagagttttg gtttttgaac |
| 961 | tagcaatgcc tgtgaaaaag aaactgaata cctaagattt ctgtcttggg gtttttggtg |
| 1021 | catgcagttg attacttctt atttttctta ccaattgtga atgttggtgt gaaacaaatt |
| 1081 | aatgaagctt ttgaatcatc cctattctgt gttttatcta gtcacataaa tggattaatt |
| 1141 | actaatttca gttgagacct tctaattggt ttttactgaa acattgaggg aacacaaatt |
| 1201 | tatgggcttc ctgatgatga ttcttctagg catcatgtcc tatagtttgt catccctgat |
| 1261 | gaatgtaaag ttacactgtt cacaaaggtt ttgtctcctt tccactgcta ttagtcatgg |
| 1321 | tcactctccc caaaatatta tattttttct ataaaaagaa aaaaatggaa aaaaattaca |
| 1381 | aggcaatgga aactattata aggccatttc cttttcacat tagataaatt actataaaga |
| 1441 | ctcctaatag cttttcctgt taaggcagac ccagtatgaa atggggatta ttatagcaac |
| 1501 | cattttgggg ctatatttac atgctactaa atttttataa taattgaaaa gattttaaca |
| 1561 | agtataaaaa attctcatag gaattaaatg tagtctccct gtgtcagact gctctttcat |
| 1621 | agtataactt taaatctttt cttcaacttg agtctttgaa gatagtttta attctgcttg |
| 1681 | tgacattaaa agattatttg ggccagttat agcttattag gtgttgaaga gaccaaggtt |
| 1741 | gcaaggccag gccctgtgtg aacctttgag ctttcataga gagtttcaca gcatggactg |
| 1801 | tgtccccacg gtcatccagt gttgtcatgc attggttagt caaaatgggg agggactagg |
| 1861 | gcagtttgga tagctcaaca agatacaatc tcactctgtg gtggtcctgc tgacaaatca |
| 1921 | agagcattgc ttttgtttct taagaaaaca aactcttttt taaaaattac ttttaaatat |
| 1981 | taactcaaaa gttgagattt tggggtggtg gtgtgccaag acattaattt tttttttaaa |
| 2041 | caatgaagtg aaaaagtttt acaatctcta ggtttggcta gttctcttaa cactggttaa |
| 2101 | attaacattg cataaacact tttcaagtct gatccatatt taataatgct ttaaaataaa |
| 2161 | aataaaaaca atccttttga taaatttaaa atgttactta ttttaaaata aatgaagtga |
| 2221 | gatggcatgg tgaggtgaaa gtatcactgg actaggaaga aggtgactta ggttctagat |
| 2281 | aggtgtcttt taggactctg attttgagga catcacttac tatccatttc ttcatgttaa |
| 2341 | aagaagtcat ctcaaactct tagttttttt tttttacaac tatgtaattt atattccatt |
| 2401 | tacataagga tacacttatt tgtcaagctc agcacaatct gtaaattttt aacctatgtt |
| 2461 | acaccatctt cagtgccagt cttgggcaaa attgtgcaag aggtgaagtt tatatttgaa |
| 2521 | tatccattct cgttttagga ctcttcttcc atattagtgt catcttgcct ccctaccttc |
| 2581 | cacatgcccc atgacttgat gcagttttaa tacttgtaat tcccctaacc ataagattta |
| 2641 | ctgctgctgt ggatatctcc atgaagtttt cccactgagt cacatcagaa atgccctaca |
| 2701 | tcttatttcc tcagggctca agagaatctg acagatacca taaagggatt tgacctaatc |
| 2761 | actaattttc aggtggtggc tgatgctttg aacatctctt tgctgcccaa tccattagcg |
| 2821 | acagtaggat ttttcaaacc tggtatgaat agacagaacc ctatccagtg gaaggagaat |
| 2881 | ttaataaaga tagtgctgaa agaattcctt aggtaatcta taactaggac tactcctggt |
| 2941 | aacagtaata cattccattg ttttagtaac cagaaatctt catgcaatga aaaatacttt |
| 3001 | aattcatgaa gcttactttt tttttttggt gtcagagtct cgctcttgtc acccaggctg |
| 3061 | gaatgcagtg gcgccatctc agctcactgc aacctccatc tcccaggttc aagcgattct |
| 3121 | cgtgcctcgg cctcctgagt agctgggatt acaggcgtgt gccactacac tcaactaatt |
| 3181 | tttgtatttt taggagagac ggggtttcac cctgttggcc aggctggtct cgaactcctg |
| 3241 | acctcaagtg atGcacccac cttggcctca taaacctgtt ttgcagaact catttattca |
| 3301 | gcaaatattt attgagtgcc taccagatgc cagtcaccgc acaaggcact gggtatatgg |
| 3361 | tatccccaaa caagagacat aatcccggtc cttaggtagt gctagtgtgg tctgtaatat |
| 3421 | cttactaagg cctttggtat acgacccaga gataacacga tgcgtatttt agttttgcaa |
| 3481 | agaaggggtt tggtctctgt gccagctcta taattgtttt gctacgattc cactgaaact |
| 3541 | cttcgatcaa gctactttat gtaaatcact tcattgtttt aaaggaataa acttgattat |
| 3601 | attgtttttt tatttggcat aactgtgatt cttttaggac aattactgta cacattaagg |
| 3661 | tgtatgtcag atattcatat tgacccaaat gtgtaatatt ccagttttct ctgcataagt |
| 3721 | aattaaaata tacttaaaaa ttaatagttt tatctgggta caaataaaca ggtgcctgaa |
| 3781 | ctagttcaca gacaaggaaa cttctatgta aaaatcacta tgatttctga attgctatgt |
| 3841 | gaaactacag atctttggaa cactgtttag gtagggtgtt aagacttaca cagtacctcg |
| 3901 | tttctacaca gagaaagaaa tggccatact tcaggaactg cagtgcttat gaggggatat |
| 3961 | ttaggcctct tgaatttttg atgtagatgg gcattttttt aaggtagtgg ttaattacct |
| 4021 | ttatgtgaac tttgaatggt ttaacaaaag atttgttttt gtagagattt taaaggggga |
| 4081 | gaattctaga aataaatgtt acctaattat tacagcctta aagacaaaaa tccttgttga |
| 4141 | agttttttta aaaaaagcta aattacatag acttaggcat taacatgttt gtggaagaat |
| 4201 | atagcagacg tatattgtat catttgagtg aatgttccca agtaggcatt ctaggctcta |
| 4261 | tttaactgag tcacactgca taggaattta gaacctaact tttataggtt atcaaaactg |
| 4321 | ttgtcaccat tgcacaattt tgtcctaata tatacataga aactttgtgg ggcatgttaa |
| 4381 | gttacagttt gcacaagttc atctcatttg tattccattg attttttttt tcttctaaac |
| 4441 | attttttctt caaacagtat ataacttttt ttaggggatt tttttttaga cagcaaaaac |
| 4501 | tatctgaaga tttccatttg tcaaaaagta atgatttctt gataattgtg tagtaatgtt |
| 4561 | ttttagaacc cagcagttac cttaaagctg aatttatatt tagtaacttc tgtgttaata |
| 4621 | ctggatagca tgaattctgc attgagaaac tgaatagctg tcataaaatg aaactttctt |
| 4681 | tctaaagaaa gatactcaca tgagttcttg aagaatagtc ataactagat taagatctgt |
| 4741 | gttttagttt aatagtttga agtgcctgtt tgggataatg ataggtaatt tagatgaatt |
| 4801 | taggggaaaa aaaagttatc tgcagatatg ttgagggccc atctctcccc ccacaccccc |
| 4861 | acagagctaa ctgggttaca gtgttttatc cgaaagtttc caattccact gtcttgtgtt |
| 4921 | ttcatgttga aaatactttt gcatttttcc tttgagtgcc aatttcttac tagtactatt |
| 4981 | tcttaatgta acatgtttac ctggaatgta ttttaactat ttttgtatag tgtaaactga |
| 5041 | aacatgcaca ttttgtacat tgtgctttct tttgtgggac atatgcagtg tgatccagtt |
| 5101 | gttttccatc atttggttgc gctgacctag gaatgttggt catatcaaac attaaaaatg |
| 5161 | accactcttt taattgaaat taacttttaa atgtttatag gagtatgtgc tgtgaagtga |
| 5221 | tctaaaattt gtaatatttt tgtcatgaac tgtactactc ctaattattg taatgtaata |
| 5281 | aaaatagtta cagtgacaaa aaaaaaaaaa aa |

The KRAS variant is the result of a substitution of a G for a U at position 4 of SEQ ID NO: 6 of LCS6. This KRAS variant comprises the sequence GAUGCACCCACCUUGGCCUCA (SNP bolded for emphasis) (SEQ ID NO: 13).

The KRAS variant leads to altered KRAS expression by disrupting the miRNA regulation of a KRAS. The identification and characterization of the KRAS variant is further described in International Application No. PCT/US08/65302 (WO 2008/151004), the contents of which are incorporated by reference in their entirety.

Let-7 Family miRNAs

Expression of let-7 family miRNAs is decreased in endometrial cells that carry the KRAS variant. Interestingly, the let-7 family of miRNAs binds to the let-7 complementary site in which the KRAS variant is located. The presence of the KRAS variant interferes with let-7 binding to KRAS. By interfering, the KRAS variant either induces let-7 to bind more or less tightly to LCS6 of KRAS. It was discovered that endometrial cells containing the KRAS variant have higher levels of KRAS mRNA compared to wild type cells, and increased levels of the KRAS protein. Thus, while not wishing to be bound by theory, the presence of the KRAS variant within endothelial cells may interfere with the ability of let-7 to bind to KRAS and inhibit protein translation, allowing higher KRAS protein levels.

The presence of the KRAS-variant in endometriosis is also associated with significantly lower levels of let-7 miRNAs. For instance, let-7 miRNA expression is decreased by 2-fold (2×), 3-fold (3×), 4-fold (4×), 5-fold (5×), 6-fold (6×), 7-fold (7×), 8-fold (8×), 9-fold (9×), 10-fold (10×), 20-fold (20×), 50-fold (50×), 100-fold (100×), 200-fold (200×), 500-fold (500×), 1000-fold (1000×), or any multiplier in between. Alternatively, or in addition, the statistically significant difference between the reduction of let-7 miRNA expression in a cell obtained from a subject who has endometriosis with the KRAS-variant compared to the level of let-7 miRNA expression in a cell obtained from a subject who does not have endometriosis and the KRAS-variant or endometriosis (i.e. a normal or control cell) is exemplified by a p-value of less than 0.05, preferably, a p-value of less than 0.01, or most preferably, a p-value of less than 0.001. The level of let-7 miRNA expression present in a cell obtained from a subject who has endometriosis may also be compared to a known standard level in the art. Moreover, the level of let-7 expression may be compared between an affected cell and an unaffected cell within a subject who has endometriosis, wherein the unaffected cell serves as an internal control.

Exemplary let-7 miRNAs include, but are not limited to, let-7a (let-7a-1, let-7a-2, let-7a-3), let-7b, let-7c, let-7d, let-7e, let-7f (let-7f-1 and let-7f-2), let-7g, and let-7i. For the following sequences, thymine (T) may be substituted for uracil (U). let-7a comprises the sequence UUGAUAUGUUGGAUGAUGGAGU (SEQ ID NO: 14). let-7b comprises the sequence UUGGUGUGUUGGAUGAUGGAGU (SEQ ID NO: 15). let-7c comprises the sequence UUGGUAUGUUGGAUGAUGGAGU (SEQ ID NO: 16). let-7d comprises the sequence UGAUACGUUGGAUGAUGGAGA (SEQ ID NO: 17). let-7e comprises the sequence UAUAUGUUGGAGGAUGGAGU (SEQ ID NO: 18). let-7f comprises the sequence UUGAUAUGUUAGAUGAUGGAGU (SEQ ID NO: 19). let-7g comprises the sequence GACAUGUUUGAUGAUGGAGU (SEQ ID NO: 20). let-7i comprises the sequence UGUCGUGUUUGUUGAUGGAGU (SEQ ID NO: 21).

Sequences of additional let-7 family members are publicly available from miRBase at (www.mirbase.org).

### Therapeutic Methods

Identification of the KRAS variant mutation indicates an increases risk of developing endometriosis. “Risk” in the context of the present invention, relates to the probability that an event will occur over a specific time period, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a primary tumor to a metastatic tumor or to one at risk of developing a metastatic, or from at risk of a primary metastatic event to a secondary metastatic event or from at risk of a developing a primary tumor of one type to developing a one or more primary tumors of a different type. Risk evaluation can also comprise prediction of future clinical parameters, traditional laboratory risk factor values, or other indices of cancer, either in absolute or relative terms in reference to a previously measured population.

An “increased risk” is meant to describe an increased probably that an individual who carries the KRAS variant will develop or has developed endometriosis, when compared to an individual who does not carry the KRAS variant. In certain embodiments, a KRAS Variant carrier is 1.5×, 2×, 2.5×, 3×, 3.5×, 4×, 4.5×, 5×, 5.5×, 6×, 6.5×, 7×, 7.5×, 8×, 8.5×, 9×, 9.5×, 10×, 20×, 30×, 40×, 50×, 60×, 70×, 80×, 90×, or 100× more likely to develop or have endometriosis than an individual who does not carry the KRAS variant.

Moreover, an “increased risk” is meant to describe an increased probably that an individual who carries the KRAS variant and has developed endometriosis, will develop or has developed ovarian cancer, when compared to an individual who does not carry the KRAS variant and does not have endometriosis. In certain embodiments, a KRAS Variant carrier with endometriosis is 1.5×, 2×, 2.5×, 3×, 3.5×, 4×, 4.5×, 5×, 5.5×, 6×, 6.5×, 7×, 7.5×, 8×, 8.5×, 9×, 9.5×, 10×, 20×, 30×, 40×, 50×, 60×, 70×, 80×, 90×, or 100× more likely to also develop or have ovarian cancer than an individual who does not carry the KRAS variant and have endometriosis.

By poor prognosis is meant that the probability of the individual surviving the development of particularly aggressive, high-risk, severe, or inherited form of endometriosis is decreased compared to the probability of surviving a less aggressive, low-risk, or mild form of endometriosis form of endometriosis. Alternatively, or in addition, poor prognosis is meant that the probability of the individual surviving the development of endomentriosis or a particularly aggressive, high-risk, severe, or inherited form of endometriosis, which may further progress into the development of ovarian cancer, is decreased compared to the probability of surviving a less aggressive, low-risk, or mild form of endometriosis or endometriosis in the absence of ovarian cancer. The ovarian cancer may be a low- or high-risk subtype of ovarian cancer. Poor prognosis is also meant to describe a less satisfactory recovery, longer recovery period, more invasive or high-risk therapeutic regime, or an increased probability of reoccurrence of the endometriosis or an associated ovarian cancer. It has been shown that the KRAS variant is predicative of the occurrence of endometriosis and, furthermore, the coincidence of the KRAS variant and development of endometriosis is associated with an increased risk of developing ovarian cancer. In particular, the KRAS variant is associated with endometriomas, a form of endometriosis in which ectopic endometrial tissue grows on one or both ovaries. This form of endometriosis is correlated with the worst outcome of endometriosis, resulting in a poor prognosis for the subject.

A subject is preferably a mammal. The mammal can be a human, non-human primate, mouse, rat, dog, cat, horse, or cow, but are not limited to these examples. A subject is typically female. A subject is one who has not been previously diagnosed as having endometriosis. The subject can be one who exhibits one or more risk factors for endometriosis. Alternatively, the subject does not exhibit a risk factor for endometrosis. Endometriosis risk factors include, but are not limited to, the presence of the KRAS variant, having a first-degree relative with endometriosis, delaying childbearing, shortened menstrual cycles (e.g. a cycle of less than 27 days), menses that are abnormally long (a period lasting longer than 8 days), müllerian duct anomalies, infertility, being aged 25-44, and failing to have or practice a protective factor against development of endometriosis. Exemplary protective against the development of endometriosis, include, but not limited to, having children early, multiple pregnancies, use of low-dose oral contraceptives, and regular exercise.

The methods described herein provide for obtaining a sample from a subject. The sample can be any tissue or fluid that contains nucleic acids. Various embodiments include, but are not limited to, paraffin imbedded tissue, frozen tissue, surgical fine needle aspirations, and cells of the pleural cavity, abdominal cavity, pelvic cavity, lung, intestine (large or small), bladder, ovary, fallopian tube, broad ligament of the uterus, uterosacral ligament, cardinal ligaments, pubocerical ligament, endometrium, myometrium, perimetrium, peritoneum, uterus, or cervix. Other embodiments include fluid samples such as blood, plasma, serum, lymph fluid, ascites, serous fluid, and urine.

### SNP Genotyping Methods

The KRAS variant is a single nucleotide polymorphism that occurs within the 3′ UTR of the human KRAS gene. Linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium”. In contrast, LD refers to any non-random genetic association between allele(s) at two or more different SNP sites, which is generally due to the physical proximity of the two loci along a chromosome. LD can occur when two or more SNPs sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD.

For screening individuals for genetic disorders (e.g. prognostic or risk) purposes, if a particular SNP site is found to be useful for screening a disorder, then the skilled artisan would recognize that other SNP sites which are in LD with this SNP site would also be useful for screening the condition. Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome.

For screening applications, polymorphisms (e.g., SNPs and/or haplotypes) that are not the actual disease-causing (causative) polymorphisms, but are in LD with such causative polymorphisms, are also useful. In such instances, the genotype of the polymorphism(s) that is/are in LD with the causative polymorphism is predictive of the genotype of the causative polymorphism and, consequently, predictive of the phenotype (e.g., disease) that is influenced by the causative SNP(s). Thus, polymorphic markers that are in LD with causative polymorphisms are useful as markers, and are particularly useful when the actual causative polymorphism(s) is/are unknown.

Linkage disequilibrium in the human genome is reviewed in: Wall et al., “Haplotype blocks and linkage disequilibrium in the human genome”, Nat Rev Genet. 2003 August; 4(8):587-97; Gamer et al., “On selecting markers for association studies: patterns of linkage disequilibrium between two and three diallelic loci”, Genet Epidemiol. 2003 January; 24(1):57-67; Ardlie et al., “Patterns of linkage disequilibrium in the human genome”, Nat Rev Genet. 2002 April; 3(4):299-309 (erratum in Nat Rev Genet 2002 July; 3(7):566); and Remm et al., “High-density genotyping and linkage disequilibrium in the human genome using chromosome 22 as a model”; Curr Opin Chem Biol. 2002 February; 6(1):24-30.

The screening techniques of the present invention may employ a variety of methodologies to determine whether a test subject has a SNP or a SNP pattern associated with an increased or decreased risk of developing a detectable trait or whether the individual suffers from a detectable trait as a result of a particular polymorphism/mutation, including, for example, methods which enable the analysis of individual chromosomes for haplotyping, family studies, single sperm DNA analysis, or somatic hybrids. The trait analyzed using the diagnostics of the invention may be any detectable trait that is commonly observed in pathologies and disorders.

The process of determining which specific nucleotide (i.e., allele) is present at each of one or more SNP positions, such as a SNP position in a nucleic acid molecule disclosed in SEQ ID NO: 11, 12 or 13, is referred to as SNP genotyping. The present invention provides methods of SNP genotyping, such as for use in screening for a variety of disorders, or determining predisposition thereto, or determining responsiveness to a form of treatment, or prognosis, or in genome mapping or SNP association analysis, etc.

Nucleic acid samples can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format. Exemplary SNP genotyping methods are described in Chen et al., “Single nucleotide polymorphism genotyping: biochemistry, protocol, cost and throughput”, Pharmacogenomics J. 2003; 3(2):77-96; Kwok et al., “Detection of single nucleotide polymorphisms”, Curr Issues Mol. Biol. 2003 April; 5(2):43-60; Shi, “Technologies for individual genotyping: detection of genetic polymorphisms in drug targets and disease genes”, Am J Pharmacogenomics. 2002; 2(3):197-205; and Kwok, “Methods for genotyping single nucleotide polymorphisms”, Annu Rev Genomics Hum Genet 2001; 2:235-58. Exemplary techniques for high-throughput SNP genotyping are described in Marnellos, “High-throughput SNP analysis for genetic association studies”, Curr Opin Drug Discov Devel. 2003 May; 6(3):317-21. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

Various methods for detecting polymorphisms include, but are not limited to, methods in which protection from cleavage agents is used to detect mismatched bases in RNA/RNA or RNA/DNA duplexes (Myers et al., Science 230:1242 (1985); Cotton et al., PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285:125-144 (1993); and Hayashi et al., Genet. Anal. Tech. Appl. 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Sequence variations at specific locations can also be assessed by nuclease protection assays such as RNase and SI protection or chemical cleavage methods.

In a preferred embodiment, SNP genotyping is performed using the TaqMan assay, which is also known as the 5′ nuclease assay (U.S. Pat. Nos. 5,210,015 and 5,538,848). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the present invention are useful in prognostic assays for a variety of disorders including cancer, and can be readily incorporated into a kit format. The present invention also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

The identity of polymorphisms may also be determined using a mismatch detection technique, including but not limited to the RNase protection method using riboprobes (Winter et al., Proc. Natl. Acad Sci. USA 82:7575, 1985; Meyers et al., Science 230:1242, 1985) and proteins which recognize nucleotide mismatches, such as the E. coli mutS protein (Modrich, P. Ann. Rev. Genet. 25:229-253, 1991). Alternatively, variant alleles can be identified by single strand conformation polymorphism (SSCP) analysis (Orita et al., Genomics 5:874-879, 1989; Humphries et al., in Molecular Diagnosis of Genetic Diseases, R. Elles, ed., pp. 321-340, 1996) or denaturing gradient gel electrophoresis (DGGE) (Wartell et al., Nuci. Acids Res. 18:2699-2706, 1990; Sheffield et al., Proc. Natl. Acad. Sci. USA 86:232-236, 1989).

A polymerase-mediated primer extension method may also be used to identify the polymorphism(s). Several such methods have been described in the patent and scientific literature and include the “Genetic Bit Analysis” method (WO92/15712) and the ligase/polymerase mediated genetic bit analysis (U.S. Pat. No. 5,679,524). Related methods are disclosed in WO91/02087, WO90/09455, WO95/17676, U.S. Pat. Nos. 5,302,509, and 5,945,283. Extended primers containing a polymorphism may be detected by mass spectrometry as described in U.S. Pat. No. 5,605,798. Another primer extension method is allele-specific PCR (Ruano et al., Nucl. Acids Res. 17:8392, 1989; Ruano et al., Nucl. Acids Res. 19, 6877-6882, 1991; WO 93/22456; Turki et al., J Clin. Invest. 95:1635-1641, 1995). In addition, multiple polymorphic sites may be investigated by simultaneously amplifying multiple regions of the nucleic acid using sets of allele-specific primers as described in Wallace et al. (WO89/10414).

Another preferred method for genotyping the KRAS variant is the use of two oligonucleotide probes in an OLA (see, e.g., U.S. Pat. No. 4,988,617). In this method, one probe hybridizes to a segment of a target nucleic acid with its 3′ most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

The following patents, patent applications, and published international patent applications, which are all hereby incorporated by reference, provide additional information pertaining to techniques for carrying out various types of OLA: U.S. Pat. Nos. 6,027,889, 6,268,148, 5494810, 5830711, and 6054564 describe OLA strategies for performing SNP detection; WO 97/31256 and WO 00/56927 describe OLA strategies for performing SNP detection using universal arrays, wherein a zipcode sequence can be introduced into one of the hybridization probes, and the resulting product, or amplified product, hybridized to a universal zip code array; U.S. application US01/17329 (and Ser. No. 09/584,905) describes OLA (or LDR) followed by PCR, wherein zipcodes are incorporated into OLA probes, and amplified PCR products are determined by electrophoretic or universal zipcode array readout; U.S. application 60/427,818, 60/445,636, and 60/445,494 describe SNPlex methods and software for multiplexed SNP detection using OLA followed by PCR, wherein zipcodes are incorporated into OLA probes, and amplified PCR products are hybridized with a zipchute reagent, and the identity of the SNP determined from electrophoretic readout of the zipchute. In some embodiments, OLA is carried out prior to PCR (or another method of nucleic acid amplification). In other embodiments, PCR (or another method of nucleic acid amplification) is carried out prior to OLA.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization—Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

Typically, the primer extension assay involves designing and annealing a primer to a template PCR amplicon upstream (5′) from a target SNP position. A mix of dideoxynucleotide triphosphates (ddNTPs) and/or deoxynucleotide triphosphates (dNTPs) are added to a reaction mixture containing template (e.g., a SNP-containing nucleic acid molecule which has typically been amplified, such as by PCR), primer, and DNA polymerase. Extension of the primer terminates at the first position in the template where a nucleotide complementary to one of the ddNTPs in the mix occurs. The primer can be either immediately adjacent (i.e., the nucleotide at the 3′ end of the primer hybridizes to the nucleotide next to the target SNP site) or two or more nucleotides removed from the SNP position. If the primer is several nucleotides removed from the target SNP position, the only limitation is that the template sequence between the 3′ end of the primer and the SNP position cannot contain a nucleotide of the same type as the one to be detected, or this will cause premature termination of the extension primer. Alternatively, if all four ddNTPs alone, with no dNTPs, are added to the reaction mixture, the primer will always be extended by only one nucleotide, corresponding to the target SNP position. In this instance, primers are designed to bind one nucleotide upstream from the SNP position (i.e., the nucleotide at the 3′ end of the primer hybridizes to the nucleotide that is immediately adjacent to the target SNP site on the 5′ side of the target SNP site). Extension by only one nucleotide is preferable, as it minimizes the overall mass of the extended primer, thereby increasing the resolution of mass differences between alternative SNP nucleotides. Furthermore, mass-tagged ddNTPs can be employed in the primer extension reactions in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer.

The extended primers can then be purified and analyzed by MALDI-TOF mass spectrometry to determine the identity of the nucleotide present at the target SNP position. In one method of analysis, the products from the primer extension reaction are combined with light absorbing crystals that form a matrix. The matrix is then hit with an energy source such as a laser to ionize and desorb the nucleic acid molecules into the gas-phase. The ionized molecules are then ejected into a flight tube and accelerated down the tube towards a detector. The time between the ionization event, such as a laser pulse, and collision of the molecule with the detector is the time of flight of that molecule. The time of flight is precisely correlated with the mass-to-charge ratio (m/z) of the ionized molecule. Ions with smaller m/z travel down the tube faster than ions with larger m/z and therefore the lighter ions reach the detector before the heavier ions. The time-of-flight is then converted into a corresponding, and highly precise, m/z. In this manner, SNPs can be identified based on the slight differences in mass, and the corresponding time of flight differences, inherent in nucleic acid molecules having different nucleotides at a single base position. For further information regarding the use of primer extension assays in conjunction with MALDI-TOF mass spectrometry for SNP genotyping, see, e.g., Wise et al., “A standard protocol for single nucleotide primer extension in the human genome using matrix-assisted laser desorption/ionization time-of-flight mass spectrometry”, Rapid Commun Mass Spectrom. 2003; 17(11):1195-202.

The following references provide further information describing mass spectrometry-based methods for SNP genotyping: Bocker, “SNP and mutation discovery using base-specific cleavage and MALDI-TOF mass spectrometry”, Bioinformatics. 2003 July; 19 Suppl 1:144-153; Storm et al., “MALDI-TOF mass spectrometry-based SNP genotyping”, Methods Mol. Biol. 2003; 212:241-62; Jurinke et al., “The use of MassARRAY technology for high throughput genotyping”, Adv Biochem Eng Biotechnol. 2002; 77:57-74; and Jurinke et al., “Automated genotyping using the DNA MassArray technology”, Methods Mol. Biol. 2002; 187:179-92.

SNPs can also be scored by direct DNA sequencing. A variety of automated sequencing procedures can be utilized ((1995) Biotechniques 19:448), including sequencing by mass spectrometry (see, e.g., PCT International Publication No. WO94/16101; Cohen et al., Adv. Chromatogr. 36:127-162 (1996); and Griffin et al., Appl. Biochem. Biotechnol. 38:147-159 (1993)). The nucleic acid sequences of the present invention enable one of ordinary skill in the art to readily design sequencing primers for such automated sequencing procedures. Commercial instrumentation, such as the Applied Biosystems 377, 3100, 3700, 3730, and 3730.times.1 DNA Analyzers (Foster City, Calif.), is commonly used in the art for automated sequencing.

Other methods that can be used to genotype the KRAS variant include single-strand conformational polymorphism (SSCP), and denaturing gradient gel electrophoresis (DGGE) (Myers et al., Nature 313:495 (1985)). SSCP identifies base differences by alteration in electrophoretic migration of single stranded PCR products, as described in Orita et al., Proc. Nat. Acad. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel (Erlich, ed., PCR Technology, Principles and Applications for DNA Amplification, W. H. Freeman and Co, New York, 1992, Chapter 7).

Sequence-specific ribozymes (U.S. Pat. No. 5,498,531) can also be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis

SNP genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

### EXAMPLES

### Example 1

### Prevalence of the KRAS Variation in Severe Endometrosis

Individuals with endometriomas were studied. Endometrioma, also referred to as an endometriod cyst, is a marker of severe endometriosis in which a lesion, cyst, or growth forms on one or both of the ovaries. Of the total number of individuals tested, a mean percentage of 31% carried the KRAS variant.

Women with peritoneal endometriosis also have an increased incidence of the mutation. Peritoneal endometriosis is considered a mild form of endometriosis, and is associated with estrogen exposure, which might be a driving force of KRAS-variant associated endometriosis. These subjects have an even higher incidence of the KRAS variant.

### Example 2

### Characterization of Endometriomal Cells with the KRAS Variant

Endometrial cells harvested from patients who carry the KRAS variant proliferate more rapidly, show increased invasion, and demonstrate increased migration than similar cells without the mutation in in vitro assays.

These endometrial cells contain increased levels of the KRAS protein and increased KRAS mRNA expression. All isoforms of let-7a (let-7a-1, let-7a-2, and let-7a-3) were decreased in endometrial cells with the mutation, compared to those without the mutation.

### Example 3

### Characterization of Response to Treatment of Endometriomal Cells with the KRAS Variant in Vivo

In a murine model of endometriosis (e.g. human endometrium under the kidney capsule of immunodeficient mice), both normal endometrium and endometrium harvested from individuals with the KRAS variation (mutation) formed endometrial masses. These lesions are characterized by the same classic endometrial glands and stroma found in typical endometriosis and in normal endometrium.

Mice containing both the normal and mutant (KRAS variant) endometrial masses are treated with progestin therapy. The responses of the normal and mutant endometrial masses are compared. Those with KRAS variant have lower levels of progesterone receptor, which predicts a poor response to progesterone and other traditional treatment.

### Example 4

### KRAS Variant and Endometriosis

### Methods

Subjects and Sample Collection.

From 2008 through 2010 a total of 150 DNA samples were collected from subjects diagnosed with endometriosis (ovarian or peritoneal) 132 of which were tested for the presence of KRAS variant allele (KV). DNA samples were received from subjects recruited at either Ponce School of Medicine and Health Sciences (PSMHS), Ponce, Puerto Rico (n=48) or the Yale University School of Medicine (n=102). The study included the women with current diagnosis or history of endometrioma (n=89) and/or peritoneal endometriosis (n=43). In all cases the diagnosis was made by biopsy; the lesion was surgically excised and the diagnosis of endometriosis was confirmed histologically. DNA was extracted from saliva, blood or tissue. Written informed consent was obtained from all participants. Approval was obtained from the Yale University School of Medicine Human Investigations Committee and from the PSMHS Institutional Review Boards as well as from Yale University IACUC for the mouse surgery protocol.

Evaluation of the LCS6 SNP in the 3′ UTR of the KRAS Gene.

DNA was isolated using the DNeasy Blood and Tissue kit or QIAamp DNA Blood Mini Kit (Qiagen) according to the manufacturer's protocol. For high-throughput genotyping, the isolated DNA samples were amplified using TaqMan PCR assays designed specifically to identify the T or G allele of the LCS6 SNP of KRAS gene (Applied Biosystems, Foster City, Calif.) as was described previously (Chin, L J, et al. Cancer Res. 2008; 68:8535-8540). Fifty nanograms (ng) of template DNA was used for each experiment. Each assay was conducted in duplicate. Positive results were confirmed by sequencing of the LCS6 region.

Tissue Collection and Cell Culture.

Endometrial biopsies were obtained from women with surgical and histological diagnosis of endometriosis. Biopsies were performed using the Pipelle catheter (CooperSurgical) both from women positive for KRAS variant allele and women with wild-type KRAS gene. As a control in the q-RT-PCR and invasion assays, endometrium from women without surgical evidence of endometriosis, but with possible other benign gynecological conditions (e.g., fibroids and benign ovarian cysts) who tested negative for the presence of the variant allele was biopsied and used as controls in these assays. Briefly, endometrium was finely minced and cells were dispersed by incubation in HBSS containing HEPES (25 mm), 1% penicillin/streptomycin, collagenase (1 mg/mL, 15 U/mg), and DNase (0.1 mg/mL, 1500 U/mg) for 60 min at 37° C. with agitation and pipetting. Endometrial cells were pelleted, washed, and suspended in Ham's F12:DMEM (1:1) containing 10% FBS, 1% penicillin/streptomycin and 1% Amphotericin B. A mixture of endometrial cells (epithelial and stromal) was passed through a 40-μm sieve (Millipore), which allowed stromal cells to pass through while epithelial cells were retained on the sieve. Human endometrial stromal cells (hESCs) were plated into 75 cm² Falcon Tissue Culture flasks (BD Biosciences). Cultured hESCs at 3-5 passages were used for q-RT-PCR, proliferation, invasion, and reporter assays as well as in the murine model. In each assay, the number of passages was identical between the variant and the control group.

Because the stromal component of endometrium plays a crucial role in regulating endometrial homeostasis and controlling epithelial growth, hESCs were chosen for use in all cell culture experiments (Arnold J T, et al (2001) Hum Reprod 16: 836-845). Moreover, it is the defect in stromal cells which is responsible for defective estradiol metabolism in eutopic and ectopic endometrial tissue in patients with endometriosis (Cheng Y H, et al. (2007) Am J Obstet Gynecol 196: 391.e1-7). For all experiments, hESCs heterozygous for the variant allele were used and compare to either normal hESCs or hESCs from women with endometriosis and who were homozygous for WT KRAS allele.

Quantitative RT-PCR.

To assess KRAS mRNA levels, total RNA was extracted from primary cultured endometrium stromal cells (ESCs) using the QIAGEN RNeasy isolation kit (QIAGEN). The experiment included hESCs from six normal subjects, six subjects with endometriosis carrying a WT KRAS allele, and six subjects carrying the variant KRAS allele. All samples were treated with RNase-free DNase (Ambion) to remove the possibility of genomic DNA contamination. RNA samples were analyzed by spectrophotometry to determine RNA concentration. mRNA (0.5 μg) was reverse-transcribed into cDNA using the iScript cDNA Synthesis Kit at 46° C. for 40 min in a reaction mixture of 20 μl (Bio-Rad Laboratories). The resultant cDNA mixtures were stored at −20° C. Gene transcripts were amplified by real-time PCR using the Bio-Rad iCycler iQ system (Bio-Rad Laboratories). All primers were obtained from W. M. Keck Oligonucleotide Synthesis Facility, Yale University (Table 2). Real-time PCR was performed using the iQ SYBR Green Supermix Kit (Bio-Rad Laboratories). Reaction mixture included cDNA template (1 μg), forward and reverse primers, RNase-free water, and the iQSYBRGreen Supermix, for a final reaction volume of 25 μl. The thermal cycling conditions were initiated by uracil-N-glycosylase activation at 50° C. for 2 min and initial denaturation at 95° C. for 10 min, then 40 cycles at 95° C. for 15 sec and annealing at 56.5° C. for 30 sec. Melting analysis was performed by heating the reaction mixture from 74° to 99° C. at a rate of 0.2 C/sec. Threshold cycle (Cₜ) and melting curves were acquired by using the quantitation and melting curve program of the Bio-Rad iCycler iQ system (Bio-Rad Laboratories). Only data with clear and single melting peaks were taken for further data analysis. Each reaction was performed in triplicate. The mRNA level of each sample was normalized to β-Actin (ACTB) expression. Relative mRNA level was presented using the formula 2^{-ΔCt}.

For miRNA detection total RNA was extracted using TRIzol (Invitrogen). A Poly (A) RT-PCR method was employed using Invitrogen NCode miRNA First-Strand cDNA Synthesis MIRC-50 kit following (Invitrogen). Conventional RT-PCR was used to assay miRNA expression with the specific forward primers to let-7a, 7b, 7c, 7d, 7e, 7f, 7g and the universal reverse primer complementary to the anchor primer. Anchor RT primer was used as the template for negative control and U6 small nuclear RNA was used as a control to determine relative miRNA expression. The real-time PCR profile was the same as described above, except for a melting temperature of 59° C.

TABLE 2

| Primers | Sequence | SEQ ID NO: |
|---|---|---|
| KRAS forward | tgaggactggggagggcttt | 22 |
| KRAS reverse | aggcatcatcaacaccctgtct | 23 |
| ACTB forward | gaagatcaagatcattgctc | 24 |
| ACTB reverse | aacgcaactaagtcatagtc | 25 |
| U6 forward | ctcgcttcggcagcaca | 26 |
| U6 reverse | aacgcttcacgaatttgcgt | 27 |
| Let-7a | tgaggtagtaggttgtatagtt | 28 |
| Let-7b | tgaggtagtaggttgtgtggtt | 29 |
| Let-7c | tgaggtagtaggttgtatggtt | 30 |
| Let-7d | agaggtagtaggttgcatagtt | 31 |
| Let-7e | tgaggtaggaggttgtatagtt | 32 |
| Let-7f | tgaggtagtagattgtatagtt | 33 |
| Let-7g | tgaggtagtttgtacagtt | 34 |

Western Blot Analysis.

Cultured endometrial stromal cells from five women with endometriosis carrying WT KRAS and five women with endometriosis with variant KRAS were lysed in Cell Lysis Buffer (Cell Signaling Technologies), centrifuged at 12,000 rpm for 2 min at 4° C., and the supernatant was collected. The protein content was quantified by the BCA assay method by using a protein assay kit (Bio-Rad Laboratories). Aliquots (20 μg) were loaded onto 4-20% gradient polyacrylamide gel in MOPS buffer system (Invitrogen) and transferred to a nitrocellulose membrane by using a Transblot apparatus (Bio-Rad Laboratories) at 35 volts (V) overnight at 4° C. Subsequently, the membranes were incubated in blocking buffer (5% milk) for 1 hr and then immunoblotted with mouse monoclonal KRAS antibody (1:200, ab55391, Abcam) and Pan Actin antibody (1:25,000, 4968, Cell Signaling Technologies) overnight. After incubation with the primary antibody, the membranes were washed three times for 15 mM with TBS [10 mM Tris-HCl (pH 7.4), 0.5 M NaCl] plus Tween 20 (0.2% vol/vol; TBST) and were incubated for 2 hours (h) in the corresponding horseradish peroxidase (HRP)-conjugated secondary antibody (1:2000) (Invitrogen). The membranes were washed three times for 5 mM in TBST. Proteins were detected with enhanced chemiluminescence (PerkinElmer). Quantification was performed using the ImageJ program.

Transfection and Luciferase Assay.

pGL3 derivatives containing most of the KRAS 3′ UTR and the KRAS variant LCS6 were created as follows. KRAS WT includes 3910 bp of the KRAS 3′UTR, which was amplified from human genomic DNA using the forward primer SMJ104 (ctagctagcatacaatttgtacttttttcttaaggcatac (SEQ ID NO: 35)) and reverse primer LCJ5 (ctagctagctcaatgcagaattcatgctatccag (SEQ ID NO: 36)). NheI restriction sites were included on the 5′-ends of the primers for convenient cloning. The product was first cloned into the TOPO cloning vector (Invitrogen) and then subcloned into pGL3 (Ambion) for use in subsequent luciferase assays. The luciferase reporter with the variant LCS6 KRAS 3′UTR (KRAS mLCS6) was constructed through site-directed mutagenesis of KRAS WT using GeneTailor (Invitrogen). Normal endometrial stromal cells were plated in 12-well plates at 60% confluency. The adherent cells were cotransfected with 1 mg of luciferase reporter with variant KRAS allele and a small interfering RNA (0.4 nM) designed to bind to the variant LCS6 KRAS allele (ggacuggaguucacuacgugu (SEQ ID NO: 37)). Qiagen AllStars Negative Control siRNA (0.4 nM) was used as negative control. To exclude the possibility that the endogenous levels of let-7 family miRNAs were not sufficient to down regulate KRAS a control set of cells was transfected with pGL3 basic derivative carrying WT regulatory region of KRAS gene. pGL3 control vector was used as positive control of transfection efficiency. All cells were co-transfected with renilla vector (50 ng) using Fugene Transfection Reagent (Roche) using a DNA/Fugene transfection reagent volume ratio of 1:3. Luciferase activity was measured after 24 h of incubation according to the manufacturer's protocol using the Dual-Glow Luciferase assay system (Promega). Firefly luciferase activity was normalized to renilla luciferase activity values for each sample. Experiments were performed three times in triplicate. The Mann-Whitney test was used for statistical analysis of the data.

Proliferation Assay.

Proliferation assay was performed with 5-bromo-2′-deoxy-uridine Labeling and Detection Kit III (Roche Applied Science). hESCs (0.5×10⁵ cells) with or without variant allele were plated into a 96-well plate. After culturing hESCs for 48 h, 10 μl of BrdU labelling solution was added into each well and incubated for 4 h. The culture medium containing the labelling solution was removed, cells were washed with serum containing wash medium, and fixed with 200 μl of precooled 0.5M ethanol in HCl for 30 min at −20° C. The cells were washed three times with serum containing medium and incubated with 100 μl of nucleases working solution per well for 30 min at room temperature in a water bath. Following three washes, 100 μl of anti-BrdU-POD, Fab fragments and working solution were added. After 30 min of incubation at room temperature, the antibody conjugate was removed and the cells were washed three times with washing buffer and incubated with 100 μl of peroxidase substrate per well. When positive samples showed a distinctive green color when compared to negative control wells. Colorimetric analysis was performed using a microplate reader at 405 nm with a reference wavelength of approximately 490 nm (Bio-Rad Laboratories). The assay was performed three times in triplicate using hESCs obtained from six different subjects in each group. The Mann-Whitney U-test was used for statistical analysis of the data.

Invasion Assays.

The invasion capacity from serum free towards serum containing medium through extracellular matrix gel and 8 μm pore membrane was analyzed using the Millipore Colorimetric Migration Assay on 24-well plates (BD Falcon). Briefly, the membrane of each insert was covered with 100 μl of ECM gel (Sigma-Aldrich) and the cells were kept in serum free medium (DMEM F12+1% penicillin/streptomycin+1% Amphotericin B) for 24 h. hESCs (2×10⁵ cells) from women without endometriosis and with endometriosis with WT or variant KRAS alleles were seeded into the inserts and incubated for 48 h. The lower chamber for this assay included 24-well tissue culture plates (BD Falcon), which contained 500 μl of DMEM/F12 (1:1) with 10% FBS, 1% penicillin/streptomycin and 1% Amphothericin B. For the control, hESCs (2×10⁵ cells) were plated directly into the lower chamber which represented 100% invasion. Invaded cells were stained, collected and lysed according to the manufacturer's instructions. Optical densities were read in triplicate at 560 nm using a Bio-Rad Laboratories plate reader. To determine the relative percent of invasion, results were compared to the 100% invasion control. Each experiment was performed three times in triplicate using specimens from six subjects without endometriosis, six subjects with endometriosis carrying WT KRAS allele and nine subjects with endometriosis carrying variant KRAS allele. The Mann-Whitney U-test was used to assess the significance of the difference in the acquired data.

Murine Endometriosis Model.

Female 6-8-week-old immune-deficient mice (CB17SCID) were purchased from Charles River Laboratory. Experimental endometriosis was created in six mice. All surgeries and tissue collection were synchronized by use of vaginal cytology. All mice were operated on in their proestrous phase. In three mice endometriosis was created using cultured endometrial stromal cells from three different subjects with endometriosis and who tested positive for the KRAS variant SNP (also known as the LCS6 SNP). In the other three mice endometriosis was created using cultured endometrial stromal cells from three different subjects with endometriosis but negative for the SNP (i.e. possessing the normal KRAS LCS6). Cells were cultured as described above, passaged 3-5 times, used at 100% confluency and harvested using 0.05% trypsin/EDTA. Cells were counted manually with a hemocytometer. The protocol for mouse kidney capsule cell transplantation was adopted and modified from Szot et al (Szot G L, et al. (2007) J Vis Exp 9: 404). Briefly, 1×10⁶ cells were suspended in 20 ml normal saline and transferred to PESO tubing system (BD Biosciences). The tubing was placed into 15 ml Falcon tubes and centrifuged at 1000 rpm to form a pellet. The tubing was maintained at 37° C. until the procedure was performed. Each mouse was anaesthetized with intraperitoneal injection of xylazine/ketamine solution (100 mg/kg and 10 mg/kg, respectively). Meloxicam was used for analgesia (0.2 mg/kg). The kidney was exteriorized and normal saline solution was injected in the peritoneal cavity to avoid dehydration. The kidney capsule was incised using a 27 gauge needle, the tubing containing the cell pellet inserted into the incision and the pellet was released under the kidney capsule. Thermocautery was used to close the kidney incision. The kidney was put back into the abdominal cavity and the abdominal wall was sutured. The skin was closed using a surgical stapler. After 4 weeks mice were sacrificed and the kidneys harvested, formalin fixed and paraffin embedded. Five micron sections were stained using haematoxylin and eosin (H&E) or used for immunohistochemistry (IHC) as described herein. The experiment protocol was approved by Yale Institutional Animal Care and Use Committee (IACUC).

Immunohistochemistry.

IHC was conducted on formalin-fixed paraffin-embedded mouse kidneys containing transplanted hESCs that had either the variant or normal LCS6 allele of the KRAS gene. Sections were deparaffinized and dehydrated through a series of xylene and ethanol washes. Each specimen was stained with H&E for histological evaluation. For IHC analysis, after a 5-min rinse in distilled water, an antigen-presenting step was performed by steaming the slides in 0.01 mole/liter sodium citrate buffer for 20 min, followed by cooling for 20 min Slides were rinsed for 5 min in PBS with 0.1% Tween 20 (PBST), and sections were circumscribed with a hydrophobic pen. Endogenous peroxidase was inactivated by incubation in 3% hydrogen peroxide for 5 min, followed by a 5-min PBST wash. After a preincubation with 2% normal goat or horse serum to block non-specific sites, sections were incubated with primary antibodies in a humidified chamber for 18 h at 4° C. Antibodies used were against PCNA, ERα, PR (Santa Cruz Biotechnology) and Cleaved Caspase-3 (Asp175; Cell Signaling Technology). Slides were then incubated with the appropriate biotin conjugated secondary antibodies followed by avitin-biotin-horseradish peroxidase complex and diaminobenzidine tetrahydrochloride before counterstaining with Gill's haematoxylin (ABC kit; Vector Laboratories). Negative control sections were processed in an identical manner but substituting primary antibodies with normal rabbit IgG. All negative control sections showed no color reaction. The number of stained nuclei was counted separately in epithelium and stroma in five high-power fields on each slide by three-independent researchers and averaged for each experimental animal. The Mann-Whitney U-test was used to determine the significance of differences between experimental groups.

Statistical Analysis.

Chi-squared test was used to compare the frequencies of clinical symptoms among the groups of patients with non-variant (WT) and alternative (or variant) KRAS allele. T-test was used to evaluate statistical significance of experiments used to asses KRAS mRNA and let-7 miRNA. The Mann-Whitney U-test was used to assess the significance of the differences in proliferation, invasion and luciferase activity and to compare IHC staining indices. Reported are mean±standard error of the mean (SEM).

### Results

Prevalence of the KRAS LCS6 Variant in Women with Endometriosis.

To determine the prevalence of the KRAS variant allele in women with endometriosis, 150 subjects were identified who provided DNA samples. Subjects were an average age of 32.9 years and endometriosis was diagnosed an average of 7 years prior to the study. Thirty-one percent had a family history of endometriosis. DNA suitable for analysis was obtained from 132 subjects. The study included women with a current diagnosis or history of endometrioma (n=89) and/or peritoneal endometriosis (n=43). Among those subjects with an endometrioma, 69% (n=61) had co-existing peritoneal endometriosis. Staging of disease was made according to the American Society for Reproductive Medicine revised classification of endometriosis (Table 1). Surgical diagnosis of severe (stage IV) and moderate (stage III) or minimal/mild (stage I/II) endometriosis was made in 56% (n=74), 33% (n=43) and 11% (n=15) of subjects, respectively. 77% (n=102) of patients had severe pain (pain which interfered with everyday activities) and/or dysmenorrhea. 27% (n=36) of subjects were diagnosed with infertility. An irregular menstrual cycle was present in 35% (n=46) of subjects.

The allele frequencies of the LCS6 SNP were determined using a collection of genomic DNA from 2433 healthy individuals from a global set of 46 populations (Chin L J, et al (2008) Cancer Res 268: 8535-8540). An extensive database of genetic variations in these samples can be found, along with the population descriptions, in ALFRED (Cheung K, et al. (2000) Nucleic Acids Res 28: 361-363). The results demonstrated that less than 3% of the 4,866 chromosomes, or 5.8% of people tested, had the G allele (variant) at the LCS6 SNP site. The frequency of this allele varied across geographic populations, with ‘European’ populations exhibiting the variant allele most frequently (7.6% of the chromosomes tested); African populations less frequently (less than 2.0% of chromosomes tested) and ‘Asian’ and Native American populations infrequently (less than 0.4% of chromosomes tested) (Chin L J, et al (2008) Cancer Res 268: 8535-8540).

Of the 132 women with endometriosis, 41 (31%) were found to carry a variant allele at LCS6 in the KRAS 3′ UTR that prevents let-7 miRNA inhibition of KRAS. In subjects with ovarian endometriomas, 23 of 89 had the variant LCS6 while 18 out of 43 women with peritoneal endometriosis carried this alternative allele (25.8 and 41.8%, respectively). 5.3% of patients were homozygous for the alternative allele (7 out of 132), which represents 17% of KRAS-variant-positive cases. There was no difference in the mean age of subjects with KRAS non-variant and variant alleles (Table 3). A total of 56 and 57% of subjects with the non-variant and variant KRAS alleles, respectively, were surgically diagnosed with severe (stage IV) endometriosis. There was no difference in the frequency of endometriomas or peritoneal endometriosis between the groups with the KRAS variant or the non-variant allele. Subjects with the alternative KRAS allele, however, more frequently had infertility (42% vs. 18% in the group with variant and non-variant KRAS allele, respectively, p=0.0033). In contrast, those with non-variant KRAS allele complained of severe pain, dysmenorrhea and dyspareunia more frequently than the group with the KRAS variant allele. Those symptoms were found in 77% (severe pain), 96% (dysmenorrhea) and 69% (dyspareunia) vs. 42% (severe pain), 42% (dysmenorrhea) and 28% (dyspareunia) of women with the non-variant and variant KRAS genes, respectively (severe pain, p=0.0001; dysmenorrhea, p=0.00001; and dyspareunia, p=0.0001; respectively). Irregular menstrual cycles were equally frequent in patients from each group. The subjects were ethnically diverse and included 66 Caucasian, 9 black and 57 hispanic subjects. The rate of the KRAS variant was not significantly different between ethnic groups.

The KRAS variant that prevents let-7 miRNA inhibition of KRAS was significantly increased in women with endometriosis. The prevalence of the KRAS variant allele in the endometriosis patient cohort is significantly higher than expected in any existing geographic population, demonstrating that this variant allele is a marker of increased endometriosis risk.

TABLE 3
Clinical Characteristics of women with the non-variant and variant
KRAS alleles.

| | Variant KRAS | Non-variant (wt) | |
|---|---|---|---|
| | allele | KRAS allele | p-Value |
| Age | 34.1 | 31.3 | 0.35 |
| Stage IV disease | 57% | 56% | 1 |
| Years from diagnosis | 9 | 6 | 0.31 |
| Infertility | 42% | 18% | 0.003 |
| Irregular menses | 28% | 35% | 0.34 |
| Severe pain | 42% | 77% | 0.001 |
| Dysmenorrhea | 42% | 96% | 0.0001 |
| Dyspareunia | 28% | 69% | 0.001 |

Subjects with the non-variant KRAS allele had severe pelvic pain, dyspareunia and dysmenorrhea more frequently than patients with the alternative KRAS allele. Subjects with the variant KRAS allele were more likely to be infertile. Mean age and severity were not different.
n = 41 subjects with KRAS variant and 91 subjects with the WT allele.

Effect of the LCS6 Variant Allele on KRAS and Let-7 Expression.

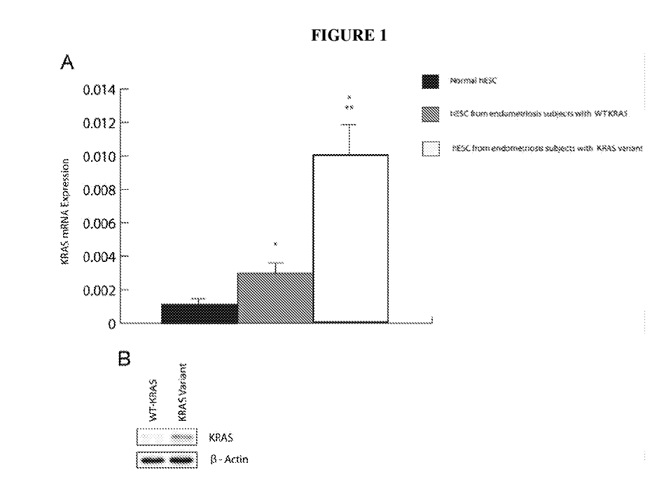

Quantitative polymerase chain reaction (after reverse transcription (RT-PCR)) was used to determine the level of KRAS mRNA in cultured hESCs from women without endometriosis, women with endometriosis heterozygous for the variant allele, and women homozygous for the wild-type (WT) allele (n=10 subjects per group). hESCs from women without endometriosis were found to express approximately threefold lower levels of KRAS mRNA compared to both hESCs from women with endometriosis carrying WT KRAS (KRAS/Actin: 0.001±0.0002 vs. 0.003±0.0004; p=0.0007) and 10-fold lower mRNA levels compared to those carrying the variant KRAS allele (0.01±0.002; p=0.0001). KRAS mRNA was approximately threefold higher in hESCs of subjects with the variant KRAS LCS6 compared to hESCs from subjects with the non-variant allele (p=0.0049; FIG. 1A). Western blot analysis showed that hESCs from women with the KRAS SNP (also known as the variant allele) had a 2.8-fold increase in KRAS protein compared to endometrium carrying the non-variant allele (FIG. 1B).

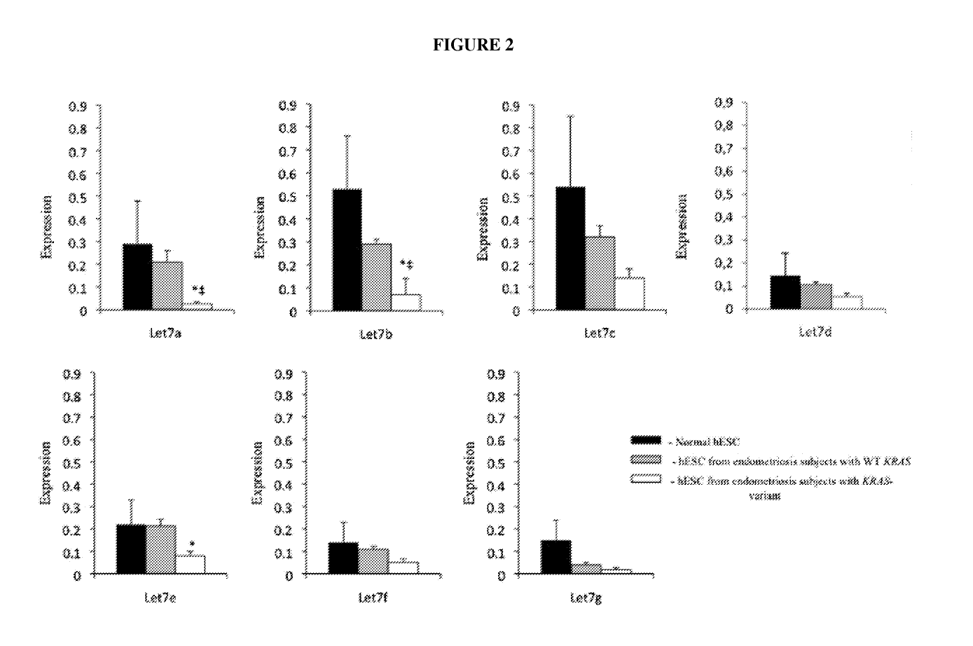

Let-7 binds to the nonvariant but not the variant LCS6 allele preventing KRAS protein synthesis. To assess the possibility of compensatory changes in let-7 miRNAs in the setting of the variant allele and elevated KRAS protein, the expression levels of let-7a-g miRNAs were determined (FIG. 2). Cultured endometrial stromal cells from women with endometriosis with the LCS6 variant in the KRAS gene showed lower levels of let-7a, let 7b and let-7e (0.26±0.009, 0.07±0.01 and 0.08±0.02, respectively), compared to hESCs from women with endometriosis with non-variant KRAS (0.21±0.05, 0.29±0.02, and 0.21±0.029, respectively) (p=0.0047, 0.003 and 0.05, respectively). Let-7a and let 7b were also lower in KRAS variant cells from endometriotic women compared to normal hESCs (0.29±0.188, 0.53±0.23, respectively) (p=0.05 and p=0.02, respectively). Compared to hESCs isolated from normal endometrium, hESCs from women with endometriosis exhibited lower levels of let-7 family miRNA. These alterations in let-7 in the presence of the KRAS variant allele could be a result of a feedback mechanism or due to another unidentified independent factor, however, these results agree with prior published findings of lower let-7 in KRAS variant-associated lesions.

Effect of the LCS6 Variant on the Expression of KRAS Using a Luciferase Reporter Assay.

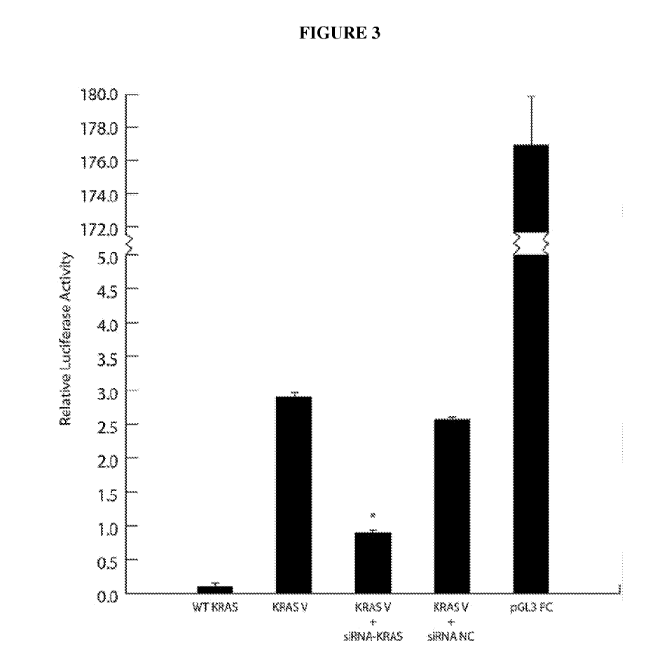

To demonstrate that the increased level of KRAS protein seen in cultured hESCs from subjects with the KRAS variant was in fact due to altered let-7 binding to the mutant LCS6 (also referred to herein as the KRAS variant, LCS6 variant, or KRAS LCS6 variant), an siRNA construct designed to rescue let-7 activity by binding to the altered LCS6 was introduced. Normal hESCs were co-transfected with a luciferase reporter construct carrying the variant LCS6, the siRNA or a siRNA negative control (FIG. 3). Luciferase activity in the cells transfected with a reporter carrying the KRAS variant allele was approximately 30-fold greater than the activity from the reporter with the WT allele (relative luciferase activity: 0.1±0.001 vs. 2.9±0.05). The siRNA targeting the LCS6 variant RNA reduced the luciferase activity in the cells with the reporter containing the KRAS LCS6 variant by 70% (to 0.9±0.13; p=0.045).

Effect of the KRAS-Variant Allele on Cell Proliferation and Invasion.

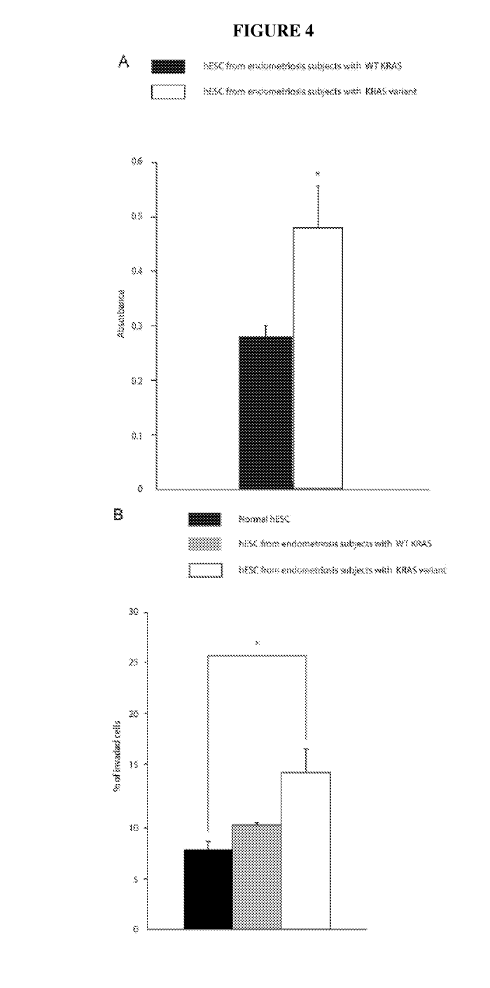

Proliferation in the presence of the KRAS LCS6 variant allele was assessed using BrdU labelling. This assay showed a 71% increase of BrdU labelling in KRAS LCS6 variant hESCs, indicative of an increased proliferation rate of endometrial cells from women with the variant allele compared to those with the nonvariant allele (absorbance 0.48±0.08 vs. 0.28±0.02; p=0.04; FIG. 4A).

To determine the invasion capacity of these cells, the percentage of cells that invaded through ECM gel was assessed. The normalized absorbance (mean absorbance in sample wells minus mean absorbance in negative control wells) was 0.089±0.006 for hESCs from control subjects, 0.118±0.019 for hESCs from subjects with endometriosis carrying WT KRAS allele and 0.175±0.026 for hESCs from subjects with endometriosis carrying variant KRAS allele. Normalized absorbance in positive control wells (100% invasion) was 1.16±0.028. The percent of invading cells was further calculated as 7±0.5, 10±1.8 and 15±2.4% in samples from women without endometriosis, women with endometriosis and non-variant KRAS LCS6 and women with endometriosis and the variant KRAS LCS6 (FIG. 4b). Invasion capacity of normal hESCs and hESCs from women with endometriosis without the variant KRAS allele were not significantly different. In contrast, the cultured endometrial cells (hESCs) with the KRAS LCS6 variant allele invaded at a significantly increased rate compared to normal hESCs (15% vs. 7%, respectively, p=0.013).

Behavior of Endometrial Cells with the KRAS LCS6 Variant Allele in a Murine Endometriosis Model.

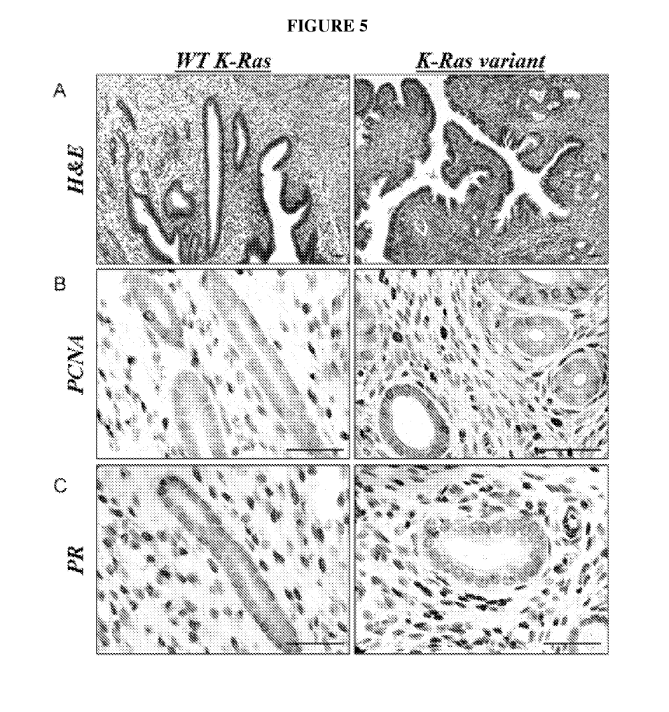

To evaluate differences in growth parameters in vivo, capacity for endometriotic lesion formation as well as histopathological and molecular characteristics of cultured endometrial stromal cells containing non-variant and variant alleles of the KRAS gene, a mouse model of endometriosis was used. hESCs obtained from subjects with and without the LCS6 variant were transplanted under the kidney capsule of immune-deficient mice. Both non-variant and LCS6 variant cells formed endometriosis-like lesions with both glandular and stromal components (FIG. 5a). The glandular component likely originated from progenitor stem cells in the culture as recently described (Cervello I, et al. (2011) PLoS ONE 6: 21221). Glandular cells are not identified in these cultures by the third passage, however, a small number of contaminants cannot be excluded (Taylor H, et al. (1998) J Clin

Invest 101: 1379-1384). Analysis of proliferation marker PCNA showed more cells (both epithelial and stromal) with stained nuclei in the lesion derived from LCS6 variant cells compared to those derived from non-variant cells. The percentage of stained nuclei in epithelium of lesions carrying variant KRAS allele was 54±5% vs. 8±1% in lesions created by non-variant hESCs (p=0.02). Stromal cells from lesions with the SNP exhibited 56±5% of nuclei staining while in the lesions with WT KRAS, the percentage of positively stained nuclei was 34±6% (p=0.043; FIG. 5b). These results suggest increased proliferation of cells harboring the variant allele and were consistent with the results of the in vitro proliferation experiment. Apoptosis was assessed using cleaved caspase 3, and the amount of cleaved caspase 3 was equivalent in lesions derived from cells with either non-variant or LCS6 variant alleles. Proliferation was increased without an increase in apoptosis in the LCS6 variant lesions.

Progestins are commonly used to treat endometriosis and progesterone resistance has been described in this disease (Bulun S E, et al. (2006) Mol Cell Endocrinol 248: 94-103; Cakmak H and Taylor H S (2010) Semin Reprod Med 28: 69-74). To determine if alterations in the expression of sex steroid receptors were seen in the presence of the variant allele, levels of estrogen receptor alpha (ERα) and progesterone receptor (PR) A and B were assessed. ERα levels were similar between lesions derived from cells with or without the variant LCS6 allele. The number of nuclei positively stained for PR was decreased in the lesions with KRAS variant allele compared to the lesions created by WT hESCs in both epithelium (35±4% vs. 75±3%, respectively, p=0.02) and stroma (13±8% vs. 78±6%, respectively, p=0.028; FIG. 5c). The KRAS LCS6 variant cells retain the receptor for estrogen, which drives proliferation in endometrial cells; however, these cells show decreased PR, which drives differentiation.

In this study, a novel gene mutation associated with endometriosis was identified. A variant SNP in the LCS6 let-7 miRNA binding site of the KRAS 3′UTR was found in 31% of all cases of endometriosis, which is significantly higher than the 5.8% incidence observed in world populations. This variant allele is, therefore, potentially contributing to nearly one third of all endometriosis cases. Subjects with the non-variant KRAS allele more commonly presented with pain and dysmenorrhea than those with the variant allele; this may be due to the higher incidence of peritoneal endometriosis (as a proportion of total endometriosis), which is more likely to be symptomatic than are ovarian endometriomas. Subjects with the KRAS variant more often presented with infertility. Stage IV endometriosis, however, was equally common among subjects in each group.

This finding provides a mechanism for the pathogenesis of endometriosis in women with this functional mutation. Loss of the normal LCS6 let-7 binding site was associated with increased transcription and translation of KRAS. Ras proteins are crucial regulators of tyrosine kinase mitogenic and oncogenic activity. Effects of Ras activation include increased cell survival and proliferation (Khosravi-Far R and Der C J (1994) Cancer Metastasis Rev 13: 67-89; Schubbert S, et al. (2007) Nat Rev Cancer 7: 295-308). This study demonstrates that the presence of the variant allele leads to a higher proliferation and a higher invasion rate in endometrial cells. These properties facilitate the invasion of endometrial cells into peritoneum and ovarian cortex. This mechanism supports the most accepted theory for the origin of endometriosis: retrograde menstruation and subsequent implantation and invasion of susceptible tissues (Giudice L C and Kao L C (2004) Lancet 364: 1789-1799). The fact that only a portion of women develop this disease despite the nearly universal occurrence of retrograde menstruation could be explained by the presence of this allele.

Moreover, in the in vivo model of endometriosis, endometrial stromal cells harbouring the variant KRAS allele demonstrated a more aggressive behavior. Cells containing the variant allele produced lesions that proliferated more and expressed lower levels of PR, which, in turn, contributes to the diminished responsiveness to progesterone treatment. The behaviour of the variant cells (i.e., those cells carrying the KRAS variant) in this model resembled those in mice with an activated KRAS gene that form endometriosis de novo (Dinulescu D M, et al. (2005) Nat Med 11: 63-70). Limitations of our model include the variability in hormone levels through the estrous cycle despite timing by vaginal cytology; however, this model also closely resembles the normal hormonal exposure seen in women. The activation of KRAS signalling through the LCS6 variant mutation explains the inability to find activating mutations in the coding regions of KRAS in humans with endometriosis. The mouse model of endometriosis can now be reconciled with the human disease, both caused by activating mutations disrupting regulation of the KRAS gene.

This variant in the let-7 binding site of KRAS has been established as a marker for predisposition to ovarian cancer (Ratner E, et al., (2010) Cancer Res 70: 6509-6515). This supports the theory that certain types of ovarian cancer may arise from endometriosis and explains the increased risk of ovarian cancer in women with endometriosis (Nezhat F, et al. (2008) Fertil Steril 90: 1559-1570). Thus, this variant SNP in the LCS6 of KRAS is an early marker of those endometriosis patients with an increased risk of ovarian cancer.

Two large genome-wide association studies (GWAS) did not identify this variant SNP in the LCS6 of KRAS in women with endometriosis. The LCS6 polymorphism is not on the Illumina chip used in the larger European/US study. In addition, these studies confirmed the diagnosis of endometriosis by review of the medical records in a small minority of the subjects. Furthermore, in these studies, endometriosis status was not determined in the control group and can be expected to be approximately 10% in reproductive aged women. In contrast, all endometriosis subjects in this study were identified prospectively at the time of surgery and were thus clinically well annotated. Although GWAS studies are important for discovery of regions of the genome important in disease, they have not always proven to be useful in validation of functional markers due to the all-inclusive approach applied for cases and controls.

The KRAS pathway presents a potential therapeutic target for treatment of endometriosis. Our results demonstrate that synthetic small RNAs complementary to the variant allele will bind the LCS6 site and reduce reporter gene expression, suggesting a possible therapy for endometriosis. siRNA has been used as a drug because of its ability to induce specific, yet transient and reversible effects (Shim M S and Kwon Y J (2010) FEBS J 277: 4814-4827).

A SNP in the let-7 miRNA binding site in 3′UTR of the KRAS gene is a marker of endometriosis risk, explains the pathogenesis of endometriosis in the subgroup of patients with the SNP, provides a novel method of early endometriosis diagnostics, ovarian cancer prevention and offers potential treatment opportunities.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of predicting the risk of developing endometriosis in a subject, comprising the steps of:
(a) obtaining a sample from the subject; and
(b) extracting an isolated DNA or RNA sequence comprising SEQ ID NO: 6, SEQ ID NO: 13, a combination thereof, or a complementary sequence thereof;
wherein the presence of SEQ ID NO: 13 in the sample indicates that the subject has an increased risk of developing endometriosis compared to an individual who does not carry a DNA sequence comprising SEQ ID NO: 13.

2. The method of claim 1, wherein the sample is a cell or a fluid.

3. The method of claim 2, wherein the cell is isolated from the pleural cavity, the abdominal cavity, the pelvic cavity, a lung, the large intestine, the small intestine, the bladder, an ovary, a fallopian tube, a ligament, the endometrium, the myometrium, the perimetrium, the peritoneum, the uterus, or the cervix of the subject.

4. The method of claim 2, wherein the fluid is saliva, sputum, whole blood, blood plasma, blood serum, lymph fluid, ascites, serous fluid, or urine collected from the subject.

5. The method of claim 1, wherein the subject has a risk factor for developing endometriosis.

6. The method of claim 5, wherein the risk factor is selected from the group consisting of a first-degree relative with endometriosis, delayed childbearing, shortened menstrual cycles, abnormally long menses, müllerian duct anomalies, infertility, aged 25-44, lacks multiple pregnancies, lack of low-dose oral contraceptive usage, and forfeit of regular exercise.

7. The method of claim 5, wherein the risk factor is infertility.

8. The method of claim 1 or 7, wherein the endometriosis is further characterized by the occurrence of endometriomas or peritioneal endometriosis.

9. The method of claim 1, wherein the subject is further at risk for developing ovarian cancer.

10. A method of predicting the risk of developing ovarian cancer in a subject who has endometriosis, comprising the steps of (a) obtaining a sample from the subject, wherein the subject has been diagnosed with endometriosis; and
(b) extracting an isolated DNA or RNA sequence comprising SEQ ID NO: 6, SEQ ID NO: 13, a combination thereof, or a complementary sequence thereof;
wherein the presence of SEQ ID NO: 13 in the sample indicates that the subject has an increased risk of developing ovarian cancer compared to an individual who does not carry a DNA sequence comprising SEQ ID NO: 13.

11. The method of claim 10, wherein the sample is a cell or a fluid.

12. The method of claim 11, wherein the cell is isolated from the pleural cavity, the abdominal cavity, the pelvic cavity, a lung, the large intestine, the small intestine, the bladder, an ovary, a fallopian tube, a ligament, the endometrium, the myometrium, the perimetrium, the peritoneum, the uterus, or the cervix of the subject.

13. The method of claim 11, wherein the fluid is saliva, sputum, whole blood, blood plasma, blood serum, lymph fluid, ascites, serous fluid, or urine from the subject.

14. The method of claim 10, wherein the endometriosis is a severe form.

15. The method of claim 10 or 14, wherein the endometriosis is further characterized by the occurrence of endometriomas or peritoneal endometriosis.

16. A method of determining the responsiveness of a subject to a form of endometriosis treatment, the method comprising assaying for the presence of a uracil or thymine to guanine transition a position 4 of LCS6 of KRAS, wherein the presence of the transition predicts whether the endometriosis is resistant or responsive to the form of treatment.

17. The method of claim 16, wherein the form of treatment is hormonal therapy.

18. The method of claim 17, wherein the hormonal therapy is estrogen, progesterone, a progestin, a testosterone derivative, a gonadotrophin releasing hormone agonist, an aromatase inhibitor or any combination thereof.

19. The method of claim 17, wherein the hormonal therapy is not progestin.

20. A method of preventing the onset of endometriosis in a subject comprising,
(a) assaying for the presence of a uracil or thymine to guanine transition a position 4 of LCS6 of KRAS, wherein the presence of the transition indicates that the subject is at an increased risk of developing endometriosis, and
(b) administering a treatment to the subject for endometriosis before the subject presents a sign or symptom of the disease,
thereby preventing the onset of endometriosis in the subject.

21. The method of claim 20, wherein the treatment is a hormonal therapy.

22. The method of claim 21, wherein the hormonal therapy is not progestin.


SEQUENCE LISTING (37 sequences)

SEQ ID NO: 1 - DNA, 25 bp, Homo sapiens
gacaguggaaguuuuuuuuuccucg

SEQ ID NO: 2 - DNA, 19 bp, Homo sapiens
auuagugucaucuugccuc

SEQ ID NO: 3 - DNA, 25 bp, Homo sapiens
aaugcccuacaucuuauuuuccuca

SEQ ID NO: 4 - DNA, 24 bp, Homo sapiens
gguucaagcgauucucgugccucg

SEQ ID NO: 5 - DNA, 24 bp, Homo sapiens
ggcugguccgaacuccugaccuca

SEQ ID NO: 6 - DNA, 21 bp, Homo sapiens
gauucacccaccuuggccuca

SEQ ID NO: 7 - DNA, 28 bp, Homo sapiens
ggguguuaagacuugacacaguaccucg

SEQ ID NO: 8 - DNA, 28 bp, Homo sapiens
agugcuuaugaggggauauuuaggccuc

SEQ ID NO: 9 - DNA, 5436 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 10 - DNA, 5312 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 11 - DNA, 5436 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgatgcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 12 - DNA, 5312 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgatgcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 13 - DNA, 21 bp, Homo sapiens
gaugcacccaccuuggccuca

SEQ ID NO: 14 - DNA, 22 bp, Homo sapiens
uugauauguuggaugauggagu

SEQ ID NO: 15 - DNA, 22 bp, Homo sapiens
uugguguguuggaugauggagu

SEQ ID NO: 16 - DNA, 22 bp, Homo sapiens
uugguauguuggaugauggagu

SEQ ID NO: 17 - DNA, 21 bp, Homo sapiens
ugauacguuggaugauggaga

SEQ ID NO: 18 - DNA, 20 bp, Homo sapiens
uauauguuggaggauggagu

SEQ ID NO: 19 - DNA, 22 bp, Homo sapiens
uugauauguuagaugauggagu

SEQ ID NO: 20 - DNA, 20 bp, Homo sapiens
gacauguuugaugauggagu

SEQ ID NO: 21 - DNA, 21 bp, Homo sapiens
ugucguguuuguugauggagu

SEQ ID NO: 22 - DNA, 20 bp, Homo sapiens
tgaggactggggagggcttt

SEQ ID NO: 23 - DNA, 22 bp, Homo sapiens
aggcatcatcaacaccctgtct

SEQ ID NO: 24 - DNA, 20 bp, Homo sapiens
gaagatcaagatcattgctc

SEQ ID NO: 25 - DNA, 20 bp, Homo sapiens
aacgcaactaagtcatagtc

SEQ ID NO: 26 - DNA, 17 bp, Homo sapiens
ctcgcttcggcagcaca

SEQ ID NO: 27 - DNA, 20 bp, Homo sapiens
aacgcttcacgaatttgcgt

SEQ ID NO: 28 - DNA, 22 bp, Homo sapiens
tgaggtagtaggttgtatagtt

SEQ ID NO: 29 - DNA, 22 bp, Homo sapiens
tgaggtagtaggttgtgtggtt

SEQ ID NO: 30 - DNA, 22 bp, Homo sapiens
tgaggtagtaggttgtatggtt

SEQ ID NO: 31 - DNA, 22 bp, Homo sapiens
agaggtagtaggttgcatagtt

SEQ ID NO: 32 - DNA, 22 bp, Homo sapiens
tgaggtaggaggttgtatagtt

SEQ ID NO: 33 - DNA, 22 bp, Homo sapiens
tgaggtagtagattgtatagtt

SEQ ID NO: 34 - DNA, 19 bp, Homo sapiens
tgaggtagtttgtacagtt

SEQ ID NO: 35 - DNA, 40 bp, Artificial Sequence
  Primer sequence
ctagctagcatacaatttgtacttttttcttaaggcatac

SEQ ID NO: 36 - DNA, 34 bp, Artificial Sequence
  Primer sequence
ctagctagctcaatgcagaattcatgctatccag

SEQ ID NO: 37 - DNA, 21 bp, Artificial Sequence
  Primer sequence
ggacuggaguucacuacgugu